(19) United States
(12) Patent Application Publication
(10) Pub. No.: US 2026/0284000 A1
(43) Pub. Date: Sep. 24, 2026

(54) COMPOSITIONS FOR ADVANCED ACNE MEDICATION

(71) Applicant: Face Reality, LLC, Danville, CA (US)

(72) Inventors: Nancy W. LEUNG, Oakland, CA (US); Irvaz HUSIC, Livermore, CA (US)

(21) Appl. No.: 19/086,797

(22) Filed: Mar. 21, 2025

(51) Int. Cl. A61K 31/327; A61K 9/06; A61K 9/107; A61K 31/045; A61K 31/728; A61K 47/02; A61K 47/10; A61K 47/32; A61K 47/36

(52) U.S. Cl. CPC A61K 31/327; A61K 9/06; A61K 9/107; A61K 31/045; A61K 31/728; A61K 47/02; A61K 47/10; A61K 47/32; A61K 47/36

(57) ABSTRACT

Provided herein, in certain aspects, are compositions comprising micronized benzoyl peroxide, sodium hyaluronate, and bisabolol for clearing acne in a subject. Also provided herein, in certain aspects, are formulations comprising micronized benzoyl peroxide, sodium hyaluronate, and bisabolol for topical application to the skin of a subject having acne that provide effective acne clearance in the subject.

### BACKGROUND

The presence of mild, moderate, or severe acne is a common occurrence in subjects who can benefit from skin care regimens using topical formulations designed to effectively improve acne conditions. Benzoyl peroxide is used in topical formulations to treat dermatologic conditions such as acne vulgaris (commonly referred to as acne). Problems associated with topical skin care using compositions containing benzoyl peroxide may include ineffectiveness of acne clearance and/or local irritation and dryness at the site of application. There is a need for improved topical formulations for use in skin care regimens to effectively clear acne in a subject seeking improved skin condition and appearance.

### SUMMARY

Described herein, in some aspects, are compositions and topical formulations comprising micronized benzoyl peroxide. In an aspect described herein are compositions comprising: i) micronized benzoyl peroxide at a concentration of between about 2.5% w/w to about 10.5% w/w; ii) sodium hyaluronate at a concentration of between about 0.001- to about 0.5%; and iii) bisabolol at a concentration of between about 0.001- to about 0.5%. In some embodiments, the composition further comprises one or more humectants. In some embodiments, the one or more humectants comprise glycerin, or ethylhexylglycerin, or a combination thereof. In some embodiments, the composition further comprises one or more preservatives. In some embodiments, the one or more preservatives comprise phenoxyethanol, or ethylhexylglycerin, or a combination thereof. In some embodiments, the composition further comprises one or more solvents. In some embodiments, the one or more solvents comprise aqua. In some embodiments, the one or more solvents further comprise propanediol. In some embodiments, the composition further comprises one or more buffering agents. In some embodiments, the one or more buffering agents comprise sodium hydroxide. In some embodiments, the composition further comprises one of more viscosity control agents. In some embodiments, the one or more viscosity control agents comprise a carbomer, ammonium acryloyldimethyltaurate/VP copolymer, or xanthan gum, or any combination thereof. In some embodiments, the one or more viscosity control agents comprise a carbomer, ammonium acryloyldimethyltaurate/VP copolymer, and xanthan gum. In some embodiments, the aqua comprises less than about 92% w/w of the composition. In some embodiments, the micronized benzoyl peroxide is at a concentration of about 2.5% w/v. In some embodiments, the micronized benzoyl peroxide is at a concentration of about 5% w/v. In some embodiments, the micronized benzoyl peroxide is at a concentration of about 10% w/v. In some embodiments, the sodium hyaluronate is at a concentration of about 0.1%. In some embodiments, the sodium hyaluronate is at a concentration of about 0.01%. In some embodiments, the bisabolol is at a concentration of about 0.1%. In some embodiments, the bisabolol is at a concentration of about 0.01%.

In an aspect described herein are acne clearance formulations comprising: i) micronized benzoyl peroxide at a concentration of between about 2.5% to about 10.5%; ii) sodium hyaluronate at a concentration of between about 0.001-0.5%; and iii) bisabolol at a concentration of between about 0.001-0.5%; wherein the acne clearance formulation is formulated as a cream, a gel, a gel-cream, a lotion, a solution, an emulsion, a suspension or a serum, for topical application to facial skin of a subject in need thereof. In some embodiments, the formulation further comprises one or more humectants. In some embodiments, the one or more humectants comprise glycerin, or ethylhexylglycerin, or a combination thereof. In some embodiments, the formulation further comprises one or more preservatives. In some embodiments, the one or more preservatives comprise phenoxyethanol, or ethylhexylglycerin, or a combination thereof. In some embodiments, the formulation further comprises one or more solvents. In some embodiments, the one or more solvents comprise aqua. In some embodiments, the one or more solvents further comprise propanediol. In some embodiments, the formulation further comprises one or more buffering agents. In some embodiments, the one or more buffering agents comprises sodium hydroxide. In some embodiments, the formulation further comprises one of more viscosity control agents. In some embodiments, the one or more viscosity control agents comprise a carbomer, ammonium acryloyldimethyltaurate/VP copolymer, or xanthan gum, or any combination thereof. In some embodiments, the one or more viscosity control agents comprise a carbomer, ammonium acryloyldimethyltaurate/VP copolymer, and xanthan gum. In some embodiments, the aqua comprises less than about 92% w/w of the formulation. In some embodiments, the micronized benzoyl peroxide is at a concentration of about 2.5% w/w. In some embodiments, the micronized benzoyl peroxide is at a concentration of about 2.625% w/w. In some embodiments, the micronized benzoyl peroxide is at a concentration of about 4.0% w/w. In some embodiments, the micronized benzoyl peroxide is at a concentration of about 5.0% w/w. In some embodiments, the micronized benzoyl peroxide is at a concentration of about 5.25% w/w. In some embodiments, the micronized benzoyl peroxide is at a concentration of about 9.0% w/w. In some embodiments, the micronized benzoyl peroxide is at a concentration of about 10.0% w/w. In some embodiments, the micronized benzoyl peroxide is at a concentration of about 10.5% w/w. In some embodiments of formulations described herein, a D₉₀ of particle size of the micronized benzoyl peroxide is between about 0.35 to 15 microns. In some embodiments of formulations described herein, a particle size of the micronized benzoyl peroxide is no greater than 25 microns. In some embodiments, the sodium hyaluronate is at a concentration of at least about 0.005%. In some embodiments, the sodium hyaluronate is at a concentration of about 0.01%. In some embodiments, the sodium hyaluronate is at a concentration of about 0.05%. In some embodiments, the sodium hyaluronate is at a concentration of about 0.1%. In some embodiments, the bisabolol is at a concentration of at least about 0.005%. In some embodiments, the bisabolol is at a concentration of at about 0.01%. In some embodiments, the bisabolol is at a concentration of at least about 0.05%. In some embodiments, the bisabolol is at a concentration of at least about 0.1%. In some embodiments, the formulation is formulated as an aqueous solution, wherein the micronized benzoyl peroxide is at a concentration of about 2.625% w/w; wherein the sodium hyaluronate is at a concentration of about 0.1%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the formulation is formulated as an aqueous solution, wherein the micronized benzoyl peroxide is at a concentration of about 2.625% w/w; wherein the sodium hyaluronate is at a concentration of about 0.01%; wherein the bisabolol is at a concentration of about 0.1%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the formulation is formulated as an aqueous solution, wherein the micronized benzoyl peroxide is at a concentration of about 5.25% w/w; wherein the sodium hyaluronate is at a concentration of about 0.01%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the formulation is formulated as an aqueous solution, wherein the micronized benzoyl peroxide is at a concentration of about 5.25% w/w; wherein the sodium hyaluronate is at a concentration of about 0.1%; wherein the bisabolol is at a concentration of about 0.1%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the formulation is formulated as an aqueous solution, wherein the micronized benzoyl peroxide is at a concentration of about 10.5% w/w; wherein the sodium hyaluronate is at a concentration of about 0.01%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the formulation is formulated as an aqueous solution, wherein the micronized benzoyl peroxide is at a concentration of about 10.5% w/w; wherein the sodium hyaluronate is at a concentration of about 0.1%; wherein the bisabolol is at a concentration of about 0.1%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen.

In an aspect described herein are compositions comprising: i) micronized benzoyl peroxide at a concentration of between about 2.5% w/w to about 10.5% w/w; ii) sodium hyaluronate at a concentration of between about 0.001- to about 0.1%; and iii) bisabolol at a concentration of between about 0.001- to about 0.5%. In some embodiments, the composition further comprises one or more humectants, wherein the one or more humectants comprise glycerin, or ethylhexylglycerin, or a combination thereof. In some embodiments, the composition further comprises one or more preservatives, wherein the one or more preservatives comprise phenoxyethanol, or ethylhexylglycerin, or a combination thereof. In some embodiments, the composition further comprises one or more solvents, wherein the one or more solvents comprises aqua, propanediol, or aqua and propanediol. In some embodiments, the composition further comprises sodium hydroxide. In some embodiments, the composition further comprises one of more viscosity control agents, wherein the one or more viscosity control agents comprise a carbomer, ammonium acryloyldimethyltaurate/VP copolymer, or xanthan gum, or any combination thereof. In some embodiments, the micronized benzoyl peroxide is at a concentration of about 2.625% w/w, of about 5.25% w/w, or about 10.5% w/w. In some embodiments, the sodium hyaluronate is at a concentration of about 0.01% of about 0.1%. In some embodiments, the bisabolol is at a concentration of about 0.01% or about 0.1%.

In an aspect described herein are acne clearance formulations comprising: i) micronized benzoyl peroxide at a concentration of between about 2.5% w/w to about 10.5% w/w; ii) sodium hyaluronate at a concentration of between about 0.001- to about 0.1%; and iii) bisabolol at a concentration of between about 0.001- to about 0.5%; wherein the acne clearance formulation is formulated as a cream, a gel, a gel-cream, a lotion, a solution, an emulsion, a suspension or a serum, for topical application to facial skin of a subject in need thereof. In some embodiments, the formulation further comprises one or more humectants, wherein the one or more humectants comprise glycerin, or ethylhexylglycerin, or a combination thereof. In some embodiments, the formulation further comprises one or more preservatives, wherein the one or more preservatives comprise phenoxyethanol, or ethylhexylglycerin, or a combination thereof. In some embodiments, the formulation further comprises one or more solvents, wherein the one or more solvents comprises aqua, propanediol, or aqua and propanediol. In some embodiments, the formulation further comprises sodium hydroxide. In some embodiments, the formulation further comprises one of more viscosity control agents, wherein the one or more viscosity control agents comprise a carbomer, ammonium acryloyldimethyltaurate/VP copolymer, or xanthan gum, or any combination thereof. In some embodiments, the micronized benzoyl peroxide is at a concentration of about 2.625% w/w, of about 5.25% w/w, or about 10.5% w/w. In some embodiments, the sodium hyaluronate is at a concentration of about 0.01% or about 0.1%. In some embodiments, the bisabolol is at a concentration of about 0.01% or about 0.1%. In some embodiments, the formulation is formulated as an aqueous solution, formulated as an aqueous solution, wherein the micronized benzoyl peroxide is at a concentration of about 2.625% w/w; wherein the sodium hyaluronate is at a concentration of about 0.1%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the formulation is formulated as an aqueous solution, wherein the micronized benzoyl peroxide is at a concentration of about 5.25% w/w; wherein the sodium hyaluronate is at a concentration of about 0.01%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the formulation is formulated as an aqueous solution, wherein the micronized benzoyl peroxide is at a concentration of about 10.5% w/w; wherein the sodium hyaluronate is at a concentration of about 0.01%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen.

In an aspect described herein are acne clearance formulations comprising: i) micronized benzoyl peroxide at a concentration of between about 2.5% w/w to about 10.5% w/w, wherein the micronized benzoyl peroxide has a particle size of no greater than about 25 microns; ii) a form of hyaluronic acid selected from hydrolyzed hyaluronic acid, sodium hyaluronate, sodium hyaluronate crosspolymer, or acetylated sodium hyaluronate, wherein the form of hyaluronic acid is at a concentration of between about 0.01- to about 0.1%; iii) bisabolol at a concentration of between about 0.01- to about 0.25%; iv) two or more humectants selected from butylene glycol, cyclomethicone, dipropylene ethylhexylglycerin, glycol, glycerin, propylene glycol, sodium lactate, sorbitol, trehalose, xylitol, or sorbitol, or any combination thereof; v) two or more solvents, vi) one or more viscosity control agents; vii) a buffering agent; and viii) a preservative.

In an aspect described herein, are compositions comprising: i) micronized benzoyl peroxide at a concentration of between about 2.5% w/w to about 10.5% w/w; ii) sodium hyaluronate at a concentration of between about 0.001- to about 0.5%; and iii) bisabolol at a concentration of between about 0.001- to about 0.5%. In some embodiments, the composition further comprises one or more humectants. In some embodiments, the one or more humectants comprise glycerin, or ethylhexylglycerin, or a combination thereof. In some embodiments, the composition further comprises one or more preservatives. In some embodiments, the one or more preservatives comprise phenoxyethanol, or ethylhexylglycerin, or a combination thereof. In some embodiments, the composition further comprises one or more solvents. In some embodiments, the one or more solvents comprise aqua. In some embodiments, the one or more solvents further comprise propanediol. In some embodiments, the composition further comprises one or more buffering agents. In some embodiments, the one or more buffering agents comprise sodium hydroxide. In some embodiments, the composition further comprises one of more viscosity control agents. In some embodiments, the one or more viscosity control agents comprise a carbomer, ammonium acryloyldimethyltaurate/VP copolymer, or xanthan gum, or any combination thereof. In some embodiments, the one or more viscosity control agents comprise a carbomer, ammonium acryloyldimethyltaurate/VP copolymer, and xanthan gum. In some embodiments, the aqua comprises less than about 92% w/w of the composition. In some embodiments, the micronized benzoyl peroxide is at a concentration of about 2.5% w/v. In some embodiments, the micronized benzoyl peroxide is at a concentration of about 5% w/v. In some embodiments, the micronized benzoyl peroxide is at a concentration of about 10% w/v. In some embodiments, the sodium hyaluronate is at a concentration of about 0.1%. In some embodiments, the sodium hyaluronate is at a concentration of about 0.01%. In some embodiments, the bisabolol is at a concentration of about 0.1%. In some embodiments, the bisabolol is at a concentration of about 0.01%.

In an aspect described herein are acne clearance formulations comprising: i) micronized benzoyl peroxide at a concentration of between about 2.5% w/w to about 10.5% w/w; ii) sodium hyaluronate at a concentration of between about 0.001- to about 0.5%; and iii) bisabolol at a concentration of between about 0.001- to about 0.5%; wherein the acne clearance formulation is formulated as a cream, gel, gel-cream, lotion, solution, emulsion, suspension or serum, for topical application to facial skin of a subject in need thereof. In some embodiments, acne clearance formulation further comprises one or more humectants. In some embodiments, the one or more humectants comprise glycerin, or ethylhexylglycerin, or a combination thereof. In some embodiments, the acne clearance formulation further comprises one or more preservatives. In some embodiments, the one or more preservatives comprise phenoxyethanol, or ethylhexylglycerin, or a combination thereof. In some embodiments, the acne clearance formulation further comprises one or more solvents. In some embodiments, the one or more solvents comprise aqua. In some embodiments, the one or more solvents further comprise propanediol. In some embodiments, the acne clearance formulation further comprises one or more buffering agents. In some embodiments, the one or more buffering agents comprises sodium hydroxide. In some embodiments, the acne clearance formulation further comprises one of more viscosity control agents. In some embodiments, the one or more viscosity control agents comprise a carbomer, ammonium acryloyldimethyltaurate/VP copolymer, or xanthan gum, or any combination thereof. In some embodiments, the one or more viscosity control agents comprise a carbomer, ammonium acryloyldimethyltaurate/VP copolymer, and xanthan gum. In some embodiments, the aqua comprises less than about 92% w/w of the formulation. In some embodiments, the micronized benzoyl peroxide is at a concentration of about 2.5% w/w. In some embodiments, the micronized benzoyl peroxide is at a concentration of about 2.625% w/w. In some embodiments, the micronized benzoyl peroxide is at a concentration of about 4.0% w/w. In some embodiments, the micronized benzoyl peroxide is at a concentration of about 5.0% w/w. In some embodiments, the micronized benzoyl peroxide is at a concentration of about 5.25% w/w. In some embodiments, the micronized benzoyl peroxide is at a concentration of about 9.0% w/w. In some embodiments, the micronized benzoyl peroxide is at a concentration of about 10.0% w/w. In some embodiments, the micronized benzoyl peroxide is at a concentration of about 10.5% w/w. In some embodiments of acne clearance formulations described herein, a Doo of particle size of the micronized benzoyl peroxide is between about 0.35 to 15 microns. In some embodiments of acne clearance formulations described herein, a particle size of the micronized benzoyl peroxide is no greater than 25 microns. In some embodiments, the sodium hyaluronate is at a concentration of at least about 0.005%. In some embodiments, the sodium hyaluronate is at a concentration of about 0.01%. In some embodiments, the sodium hyaluronate is at a concentration of about 0.05%. In some embodiments, the sodium hyaluronate is at a concentration of about 0.1%. In some embodiments, the bisabolol is at a concentration of at least about 0.005%. In some embodiments, the bisabolol is at a concentration of at about 0.01%. In some embodiments, the bisabolol is at a concentration of at least about 0.05%. In some embodiments, the bisabolol is at a concentration of at least about 0.1%. In some embodiments, the acne clearance formulation is formulated as an aqueous solution, wherein the micronized benzoyl peroxide is at a concentration of about 2.625% w/w; wherein the sodium hyaluronate is at a concentration of about 0.1%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the acne clearance formulation is formulated as an aqueous solution, wherein the micronized benzoyl peroxide is at a concentration of about 2.625% w/w; wherein the sodium hyaluronate is at a concentration of about 0.01%; wherein the bisabolol is at a concentration of about 0.1%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the acne clearance formulation is formulated as an aqueous solution, wherein the micronized benzoyl peroxide is at a concentration of about 5.25% w/w; wherein the sodium hyaluronate is at a concentration of about 0.01%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the acne clearance formulation is formulated as an aqueous solution, wherein the micronized benzoyl peroxide is at a concentration of about 5.25% w/w; wherein the sodium hyaluronate is at a concentration of about 0.1%; wherein the bisabolol is at a concentration of about 0.1%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the acne clearance formulation is formulated as an aqueous solution, wherein the micronized benzoyl peroxide is at a concentration of about 10.5% w/w; wherein the sodium hyaluronate is at a concentration of about 0.01%; wherein the bisabolol is at a concentration of about 0.01%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen. In some embodiments, the acne clearance formulation is formulated as an aqueous solution, wherein the micronized benzoyl peroxide is at a concentration of about 10.5% w/w; wherein the sodium hyaluronate is at a concentration of about 0.1%; wherein the bisabolol is at a concentration of about 0.1%; wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and wherein the formulation is effective at clearing acne or acne rosacea when topically applied as part of a skin care regimen.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

Described herein are compositions and topical formulations to improve dermatologic conditions. In particular, the disclosure pertains to topical formulations containing benzoyl peroxide for use in clearing acne in a subject. Described herein are compositions and formulations for advanced acne medication.

### Compositions and Formulations

### Anti-Acne Micronized Benzoyl Peroxide

Benzoyl peroxide (BPO) is an organic acid that is used to improve or clear acne. Benzoyl peroxide is a non-toxic, colorless and odorless crystalline solid with a molecular weight of 242.22 and a melting point of between about 103° C. and 106° C. Benzoyl peroxide is an oxidizing agent, wherein the oxidizing properties can aid in eliminating acne-causing bacteria. Benzoyl peroxide is not recommended for use in compositions or formulations with other ingredients subject to oxidation such as natural vegetable oils, certain natural gums, or certain cream gel forming polymers. Benzoyl peroxide is an effective anti-acne component in topical skin care applications. However, benzoyl peroxide has been shown to have a concentration-dependent skin irritation potential. Benzoyl peroxide is highly insoluble in water, and benzoyl peroxide crystals maintained in suspension in a topical formulation for skin care can produce local irritation when contacting the skin. Fully dissolved or solubilized benzoyl peroxide is unstable and can decompose rapidly and is thereby unsuitable as a commercial acne clearance agent. Existing benzoyl peroxide crystalline powder may require milling for at least one hour in order to prepare a BPO paste in water having crystals sufficiently fine to be of an acceptable texture for preparing topical acne clearance products. In many instances, existing BPO preparations for use in topical skin care comprises BPO crystals larger than about 25, 30, 35, 40, 45, 50, 55, 60, 65, 70, 75, 80, 85, 90, 95, or 100 microns. As the average diameter of a skin pore is between about 25-50 microns, topical BPO skin care formulations comprising these larger BPO crystals can be less effective at delivering anti-bacterial BPO within skin pores to areas having bacteria growth that contributes to acne formation and persistence, thereby making these conventional preparations less effective. Utilizing certain solvents to aid in obtaining solubilized BPO or to reduce average BPO crystal size in a solution can also lead to skin irritation from a particular solvent remaining in a topical formulation. Non-irritative BPO preparation comprising small BPO crystals are favorable in for topical BPO formulations used in clearance of acne and reduced skin inflammation. Micronization is a technique used to reduce particle size of an active ingredient within a formulation. Micronization can be an effective mechanism of efficiently delivering an active agent to the skin through topical application. Drug particles, such as benzoyl peroxide can dissolve more efficiently if they are reduced to a small size by micronization. Micronized suspensions can provide effective delivery of small particles of benzoyl peroxide to the skin upon topical application. Micronized suspensions are suspensions containing micronized benzoyl peroxide In some aspects, advanced acne medication compositions and formulations described herein work to aid in acne clearance. In some aspects, advanced acne medication compositions and formulations described herein work to prevent acne breakouts.

In an aspect, advanced acne medication compositions described herein are anti-acne compositions. In an aspect of anti-acne compositions described herein, the anti-acne composition comprises micronized benzoyl peroxide. In some embodiments, the anti-acne composition comprises micronized benzoyl peroxide comprising finely divided benzoyl peroxide, at least 95 percent of whose particles have an average particle diameter of less than about 20, 18, 15, or 12 microns. In some embodiments, the anti-acne composition comprises micronized benzoyl peroxide comprising finely divided benzoyl peroxide, at least 97 percent of whose particles have an average particle diameter of less than about 20, 18, 15, or 12 microns. In some embodiments, the anti-acne composition comprises micronized benzoyl peroxide comprising finely divided benzoyl peroxide, essentially 100 percent of whose particles have an average particle diameter (d₅₀) below 20 microns. In some embodiments, the anti-acne composition comprises micronized benzoyl peroxide comprising finely divided benzoyl peroxide, at least 99 percent of whose particles have an average particle diameter of less than about 25 microns. In some embodiments, the anti-acne composition comprises micronized benzoyl peroxide comprising finely divided benzoyl peroxide devoid of BPO particles greater than about 10 microns. In some embodiments, the anti-acne composition comprises micronized benzoyl peroxide comprising finely divided benzoyl peroxide devoid of BPO particles greater than about 5 microns. In some embodiments, the anti-acne composition comprises micronized benzoyl peroxide comprising finely divided benzoyl peroxide devoid of BPO particles greater than about 3 microns. In some embodiments, the anti-acne composition comprises micronized benzoyl peroxide having a mean particle size of about 1-2 microns. In some embodiments, the anti-acne composition comprises micronized benzoyl peroxide having at least about 5% of BPO particles of about 0.35 microns or less. In some embodiments, the anti-acne composition comprises micronized benzoyl peroxide having at least about 95% of BPO particles of about 3.5 microns or less. In some embodiments, the anti-acne composition comprises micronized benzoyl peroxide having no more than about 1% of BPO particles less than about 0.10 microns in size in order to reduce an extent of transdermal delivery. In some embodiments, the micronized BPO is formed by wet milling in the absence of a solvent. In some embodiments, the anti-acne composition comprises an aqueous-based, micronized BPO dispersion. In some embodiments, the aqueous-based, micronized BPO dispersion is in the form of a gel. In some embodiments, the composition comprises an aqueous-based, micronized BPO dispersion (of 40% BPO) in the form of a gel. In some embodiments, the micronized BPO dispersion is at a pH of between about 4.8 to about 6.6. In some embodiments, the micronized BPO efficiently penetrates skin pores to acts as an antibacterial agent in a topical composition. In some embodiments, the micronized BPO efficiently penetrates skin pores to act as an antibacterial agent against Cutibacterium acnes (C. acnes), formerly known as Propionibacterium acnes, Corynebacterium granulosum, and/or Staphylococcus epidermidis bacteria in a topical composition. In some embodiments, the average micronized BPO size allows for a smooth topical application with a potential for greater anti-acne efficacy. In some embodiments, the average micronized BPO size allows for a non-primary irritant and a non-primary skin sensitizer as an anti-acne agent with the composition. In some embodiments, milling is not required to achieve a preferred average micronized BPO size. In some embodiments, the average micronized BPO size allows for optimized bioavailability. In some embodiments, the micronized BPO in the topical composition produces an oxygen-rich environmental within skin pores that effectively eliminates bacteria associated with acne.

In an aspect, advanced acne medication formulations described herein are anti-acne formulations. In an aspect of anti-acne formulations described herein, the anti-acne formulation comprises micronized benzoyl peroxide. In some embodiments, the anti-acne formulation comprises micronized benzoyl peroxide comprising finely divided benzoyl peroxide, at least 95 percent of whose particles have an average particle diameter of less than about 20, 18, 15, or 12 microns. In some embodiments, the anti-acne formulation comprises micronized benzoyl peroxide comprising finely divided benzoyl peroxide, at least 97 percent of whose particles have an average particle diameter of less than about 20, 18, 15, or 12 microns. In some embodiments, the anti-acne formulation comprises micronized benzoyl peroxide comprising finely divided benzoyl peroxide, essentially 100 percent of whose particles have an average particle diameter (d₅₀) below 20 microns. In some embodiments, the anti-acne formulation comprises micronized benzoyl peroxide comprising finely divided benzoyl peroxide, at least 99 percent of whose particles have an average particle diameter of less than about 25 microns. In some embodiments, the anti-acne formulation comprises micronized benzoyl peroxide comprising finely divided benzoyl peroxide devoid of BPO particles greater than about 10 microns. In some embodiments, the anti-acne formulation comprises micronized benzoyl peroxide comprising finely divided benzoyl peroxide devoid of BPO particles greater than about 5 microns. In some embodiments, the anti-acne formulation comprises micronized benzoyl peroxide comprising finely divided benzoyl peroxide devoid of BPO particles greater than about 3 microns. In some embodiments, the anti-acne formulation comprises micronized benzoyl peroxide having a mean particle size of about 1-2 microns. In some embodiments, the anti-acne formulation comprises micronized benzoyl peroxide having at least about 5% of BPO particles of about 0.35 microns or less. In some embodiments, the anti-acne formulation comprises micronized benzoyl peroxide having at least about 95% of BPO particles of about 3.5 microns or less. In some embodiments, the anti-acne formulation comprises micronized benzoyl peroxide having no more than about 1% of BPO particles less than about 0.10 microns in size in order to reduce an extent of transdermal delivery. In some embodiments, the micronized BPO is formed by wet milling in the absence of a solvent. In some embodiments, the anti-acne formulation comprises an aqueous-based, micronized BPO dispersion. In some embodiments, the aqueous-based, micronized BPO dispersion is in the form of a gel. In some embodiments, the formulation comprises an aqueous-based, micronized BPO dispersion (of 40% BPO) in the form of a gel. In some embodiments, the micronized BPO dispersion is at a pH of between about 4.8 to about 6.6. In some embodiments, the micronized BPO efficiently penetrates skin pores to acts as an antibacterial agent in a topical composition. In some embodiments, the micronized BPO efficiently penetrates skin pores to act as an antibacterial agent against C. acnes), Corynebacterium granulosum, and/or Staphylococcus epidermidis bacteria in a topical formulation. In some embodiments, the average micronized BPO size allows for a smooth topical application with a potential for greater anti-acne efficacy. In some embodiments, the average micronized BPO size allows for a non-primary irritant and a non-primary skin sensitizer as an anti-acne agent with the composition. In some embodiments, milling is not required to achieve a preferred average micronized BPO size. In some embodiments, the average micronized BPO size allows for optimized bioavailability. In some embodiments, the micronized BPO in the topical formulation produces an oxygen-rich environmental within skin pores that effectively eliminates bacteria associated with acne.

In some embodiments of compositions or formulations described herein, D₉₀ (a particle size measurement that indicates the point in a particle size distribution where 90% of the total volume of the sample is contained) of micronized benzoyl peroxide particle size is between about 5 to about 25 microns. In some embodiments of compositions or formulations described herein, D₉₀ of micronized benzoyl peroxide particle size is from about 10 to about 0.5 microns. In some embodiments of compositions or formulations described herein, D₁₀ (the diameter at which 10% of particles in a sample are smaller) of micronized benzoyl peroxide particle size is from 1 to 5 microns. In some embodiments of compositions or formulations described herein, D₁₀ of micronized benzoyl peroxide particle size is from about 0.35 to about 1.35 microns. Particle size distributions within the above specified ranges allow the benzoyl peroxide to be more thoroughly and beneficially dispersed into the skin care product, thereby making it easier to distribute the benzoyl peroxide particles evenly across the area of application and to be more easily introduced into the affected pores of the skin for treatment of acne. The particle size ranges for micronized benzoyl peroxide indicated above may also result in an appropriate overall particle surface area exposure for any given application, which may result in improved bioavailability over a period of time. Extremely fine micronized benzoyl peroxide particles (in the nanoparticulate range with particle size distribution characterized by a D₉₀ of less than 0.1 microns) can lead to further skin irritation and effectively short treatment duration per application due to overly rapid dissolution kinetics and hence rapid concentration spikes. In some embodiments of compositions or formulations described herein, D₉₀ is between about 5 to about 20 microns. In some embodiments of compositions or formulations described herein, D₉₀ is between about 7 to about 15 microns. In some embodiments of compositions or formulations described herein, D₉₀ is between about 0.35 to about 15 microns. In some embodiments of compositions or formulations described herein, D₉₀ is between about 0.35 to about 10 microns. In some embodiments of compositions or formulations described herein, BPO particles size is no greater than about 25 microns. In some embodiments of compositions or formulations described herein, components are listed in % weight/volume (w/v). In some embodiments of compositions or formulations described herein, components are listed in % weight/weight (w/w). In some embodiments of compositions or formulations described herein, the micronized benzoyl peroxide is present in a concentration of between about 0.5% w/w to about 10.5% w/w. In some embodiments of compositions or formulations described herein, the micronized benzoyl peroxide is present in a concentration of between about 1.0% w/w to about 10.5% w/w. In some embodiments of compositions or formulations described herein, the micronized benzoyl peroxide is present in a concentration of between about 2.5% w/w to about 10.5% w/w. In some embodiments of compositions or formulations described herein, the micronized benzoyl peroxide is present in a concentration of between about 2.65% w/w to about 10.5% w/w. In some embodiments of compositions or formulations described herein, the micronized BPO is present in a concentration of between about 2.0% w/w to about 2.625% w/w. In some embodiments of compositions or formulations described herein, the micronized BPO is present in a concentration of between about 2.5% w/w to about 2.625% w/w. In some embodiments of compositions or formulations described herein, the micronized BPO is present in a concentration of between about 2.5% w/w to about 4.0% w/w. In some embodiments of compositions or formulations described herein, the micronized BPO is present in a concentration of between about 4.0% w/w to about 5.25% w/w. In some embodiments of compositions or formulations described herein, the micronized BPO is present in a concentration of between about 5.0% w/w to about 5.25% w/w. In some embodiments of compositions or formulations described herein, the micronized BPO is present in a concentration of between about 5.0% w/w to about 10.5% w/w. In some embodiments of compositions or formulations described herein, the micronized benzoyl peroxide is present in a concentration of between about 7.5% w/w to about 10.5% w/w. In some embodiments of compositions or formulations described herein, the micronized benzoyl peroxide is present in a concentration of between about 9.0% w/w to about 10.5% w/w. In some embodiments of compositions or formulations described herein, the micronized benzoyl peroxide is present in a concentration of between about 10.0% w/w to about 10.5% w/w. In some embodiments of compositions or formulations described herein, the micronized BPO is present in a concentration of between about 0.5% w/v to about 10.0% w/v. In some embodiments of compositions or formulations described herein, the micronized BPO is present in a concentration of between about 1.0% w/v to about 10.0% w/v. In some embodiments of compositions or formulations described herein, the micronized BPO is present in a concentration of between about 2.5% w/v to about 10.5% w/v. In some embodiments of compositions or formulations described herein, the micronized BPO is dissolved in water (aqua). In some embodiments, the dissolved micronized benzoyl peroxide at a particular concentration is added as the anti-acne component of the composition or formulation. In some embodiments of compositions or formulations described herein, the micronized BPO is in a dispersion gel at a particular concentration and added as the anti-acne component of the composition or formulation. In some embodiments, a 40% solution of micronized BPO in a dispersion gel is added as the anti-acne component of the composition or formulation. In some embodiments of compositions or formulations described herein, dissolved micronized benzoyl peroxide present with about 2.50 to about 2.625% w/w and aqua of about 3.9375% w/w of the total of the composition or formulation is added as the anti-acne component of the composition or formulation. In some embodiments of compositions or formulations described herein, dissolved micronized benzoyl peroxide present with about 2.50 to about 4.0% w/w and aqua of about 3.9375% w/w of the total of the composition or formulation is added as the anti-acne component of the composition or formulation. In some embodiments of compositions or formulations described herein, dissolved micronized benzoyl peroxide present with about 4.0 to about 5.25% w/w and aqua of about 7.875% w/w of the total of the composition or formulation is added as the anti-acne component of the composition or formulation. In some embodiments of compositions or formulations described herein, dissolved micronized benzoyl peroxide present with about 5.0 to about 5.25% w/w and aqua of about 7.875% w/w of the total of the composition or formulation is added as the anti-acne component of the composition or formulation. In some embodiments of compositions or formulations described herein, dissolved micronized benzoyl peroxide present with about 5.0 to about 10.5% w/w and aqua of about 15.75% w/w of the total of the composition or formulation is added as the anti-acne component of the composition or formulation. In some embodiments of compositions or formulations described herein, dissolved micronized benzoyl peroxide present with about 7.5 to about 10.5% w/w and aqua of about 15.75% w/w of the total of the composition or formulation is added as the anti-acne component of the composition or formulation. In some embodiments of compositions or formulations described herein, dissolved micronized benzoyl peroxide present with about 9.0 to about 10.5% w/w and aqua of about 15.75% w/w of the total of the composition or formulation is added as the anti-acne component of the composition or formulation. In some embodiments of compositions or formulations described herein, dissolved micronized benzoyl peroxide present with about 10.0 to about 10.5% w/w and aqua of about 15.75% w/w of the total of the composition or formulation is added as the anti-acne component of the composition or formulation. In some embodiments of compositions or formulations described herein, dissolved micronized benzoyl peroxide is present at a final concentration of about 2.0%. In some embodiments of compositions or formulations described herein, dissolved micronized benzoyl peroxide is present at a final concentration of about 2.5%. In some embodiments of compositions or formulations described herein, dissolved micronized benzoyl peroxide is present at a final concentration of about 2.625%. In some embodiments of compositions or formulations described herein, dissolved micronized benzoyl peroxide is present at a final concentration of about 4.0%. In some embodiments of compositions or formulations described herein, dissolved micronized benzoyl peroxide is present at a final concentration of about 5.0%. In some embodiments of compositions or formulations described herein, dissolved micronized benzoyl peroxide is present at a final concentration of about 5.25%. In some embodiments of compositions or formulations described herein, dissolved micronized benzoyl peroxide is present at a final concentration of about 7.5%. In some embodiments of compositions or formulations described herein, dissolved micronized benzoyl peroxide is present at a final concentration of about 9.0%. In some embodiments of compositions or formulations described herein, dissolved micronized benzoyl peroxide is present at a final concentration of about 10.0%. In some embodiments of compositions or formulations described herein, dissolved micronized benzoyl peroxide is present at a final concentration of about 10.5%. In some embodiments of compositions or formulations described herein, dissolved micronized benzoyl peroxide is present at a final concentration of 2.0%. In some embodiments of compositions or formulations described herein, dissolved micronized benzoyl peroxide is present at a final concentration of 2.5%. In some embodiments of compositions or formulations described herein, dissolved micronized benzoyl peroxide is present at a final concentration of 2.625%. In some embodiments of compositions or formulations described herein, dissolved micronized benzoyl peroxide is present at a final concentration of 4.0%. In some embodiments of compositions or formulations described herein, dissolved micronized benzoyl peroxide is present at a final concentration of 5.0%. In some embodiments of compositions or formulations described herein, dissolved micronized benzoyl peroxide is present at a final concentration of 5.25%. In some embodiments of compositions or formulations described herein, dissolved micronized benzoyl peroxide is present at a final concentration of 7.5%. In some embodiments of compositions or formulations described herein, dissolved micronized benzoyl peroxide is present at a final concentration of 9.0%. In some embodiments of compositions or formulations described herein, dissolved micronized benzoyl peroxide is present at a final concentration of 10.0%. In some embodiments of compositions or formulations described herein, dissolved micronized benzoyl peroxide is present at a final concentration of 10.5%. In some embodiments, the micronized benzoyl peroxide is synthetic.

### Solvents

In an aspect of anti-acne compositions described herein, the anti-acne composition comprises a solvent in which to dissolve and/or dilute components of the composition. In an aspect of anti-acne formulations described herein, the anti-acne formulation comprises a solvent in which to dissolve and/or dilute components of the formulation. In some embodiments of compositions or formulations described herein, aqua is the solvent. In some embodiments of compositions or formulations described herein, aqua is the diluent. In some embodiments, aqua serving as the solvent and/or diluent is present between the range of about 50.0% w/w to about 90.0% w/w. In some embodiments, aqua serving as the solvent and/or diluent is present at least about 50.0% w/w. In some embodiments, aqua serving as the solvent and/or diluent is present at least about 65.0% w/w. In some embodiments, aqua serving as the solvent and/or diluent is present at least about 66.73% w/w. In some embodiments, aqua serving as the solvent and/or diluent is present at least about 70.0% w/w. In some embodiments, aqua serving as the solvent and/or diluent is present at least about 75.0% w/w. In some embodiments, aqua serving as the solvent and/or diluent is present at least about 80.355% w/w. In some embodiments, aqua serving as the solvent and/or diluent is present at least about 85.0% w/w. In some embodiments, aqua serving as the solvent and/or diluent is present at least about 86.8175% w/w. In some embodiments, aqua serving as the solvent and/or diluent is present less than about 92.0% w/w. In some embodiments, the total concentration of aqua in the composition or formulation comprises an added amount of aqua in which one or more components of the composition or formulation are dissolved. In some embodiments, the total concentration of aqua in the composition or formulation comprises an added amount of aqua in which two or more, three or more, four or more, five or more, six or more, seven or more, eight or more, or nine or more components of the composition or formulation are dissolved. In some embodiments, the added amount of aqua in which one or more components of the composition or formulation are dissolved is about 4.5865% w/w. In some embodiments, the added amount of aqua in which one or more components of the composition or formulation are dissolved is about 8.284% w/w. In some embodiments, the added amount of aqua in which one or more components of the composition or formulation are dissolved is about 16.399% w/w. In some embodiments, the total amount of aqua in the composition or formulation is at least about 83.0% w/w. In some embodiments, the total amount of aqua in the composition or formulation is at least about 88.0% w/w. In some embodiments, the total amount of aqua in the composition or formulation is at least about 91.0% w/w. In some embodiments, the total amount of aqua in the composition or formulation is about 83.129% w/w. In some embodiments, the total amount of aqua in the composition or formulation is about 88.639% w/w. In some embodiments, the total amount of aqua in the composition or formulation is about 91.404% w/w. In some embodiments of compositions or formulations described herein, propanediol is an additional solvent to aqua. In some embodiments, the propanediol is derived from a plant. In some embodiments of compositions or formulations described herein, propanediol is present in a concentration at least about 0.5% w/w. In some embodiments of compositions or formulations described herein, propanediol is present in a concentration at least about 1.0% w/w. In some embodiments of compositions or formulations described herein, propanediol is present in a concentration less than about 1.25% w/w. In some embodiments of compositions or formulations described herein, propanediol is present in a concentration of about 0.75% w/w. In some embodiments of compositions or formulations described herein, propanediol is present in a concentration of about 1.0% w/w. In some embodiments of compositions or formulations described herein, propanediol is present in a concentration of about 1.2% w/w. In some embodiments, the solvent comprises aqua and propanediol. In some embodiments, the solvent comprises aqua less than about 92.0% w/w of the formulation and propanediol about 1.0% w/w of the formulation. In some embodiments, the solvent comprises aqua at about 91.404% w/w of the formulation and propanediol about 1.0% w/w of the formulation. In some embodiments, the solvent comprises aqua at about 88.639% w/w of the formulation and propanediol about 1.0% w/w of the formulation. In some embodiments, the solvent comprises aqua at about 83.129% w/w of the formulation and propanediol about 1.0% w/w of the formulation. In some embodiments, propanediol is used as a solvent to aid in dissolving phenoxyethanol and ethylhexylglycerin.

### Humectants

In some embodiments of compositions or formulations described herein, a moisturizing agent suitable for topical application is present in the composition or formulation, wherein the moisturizing agent functions as a humectant, solvent, thickener, penetration enhancer, or buffering agent, or any combination thereof. In some embodiments, the moisturizing agent for topical application functions as a humectant. Humectants are substances that attract water from the air or from deeper in the skin. Humectants can attract and retain the moisture in the air nearby via absorption, drawing the water vapor into or beneath the skin surface of a subject. Humectants are useful for adding hydration without adding a heavy or oily feeling upon application of a composition or formulation. In some embodiments of compositions or formulations described herein, one or more humectants are included in the composition or formulation. In some embodiments of compositions or formulations described herein, two or more, three or more, four or more, five or more, six or more, seven or more, eight or more, or nine or more humectants are included in the composition or formulation. In some embodiments, the one or more humectants comprise butylene glycol, cyclomethicone, dipropylene glycol, glycerin, propylene glycol, hyaluronic acid, sodium hyaluronate, hydrolyzed hyaluronic acid, acetylated sodium hyaluronate, sodium hyaluronate crosspolymer, sodium lactate, sorbitol, trehalose, xylitol, or sorbitol, or any combination thereof. In some embodiments, the two or more, three or more, four or more, five or more, six or more, seven or more, eight or more, or nine or more humectants comprise butylene glycol, cyclomethicone, dipropylene glycol, glycerin, propylene glycol, hyaluronic acid, sodium hyaluronate, hydrolyzed hyaluronic acid, acetylated sodium hyaluronate, sodium hyaluronate crosspolymer, sodium lactate, sorbitol, trehalose, xylitol, or sorbitol, or any combination thereof. In some embodiments, the one or more humectants comprise at least about 1.0, 1.9, 2.0, 2, 49, 2.5, 2.99, 3.01, or 3.091% w/w of the composition or formulation. In some embodiments, the two or more, three or more, four or more, five or more, six or more, seven or more, eight or more, or nine or more humectants comprise at least about 1.0, 1.9, 2.0, 2, 49, 2.5, 2.99, 3.01, or 3.091% w/w of the composition or formulation. In some embodiments, the one or more humectants comprise less than about 3.091, 3.01, 3.00, 2.99 or 2.991% w/w of the composition or formulation. In some embodiments, the two or more, three or more, four or more, five or more, six or more, seven or more, eight or more, or nine or more humectants comprise less than about 3.091, 3.01, 3.00, 2.99 or 2.991% w/w of the composition or formulation. In some embodiments, the one or more humectants are mixed with a solvent before being added to the formulation. In some embodiments, the one or more humectants comprise glycerin. In some embodiments, the two or more, three or more, four or more, five or more, six or more, seven or more, eight or more, or nine or more humectants comprise glycerin. In some embodiments, the one or more humectants consists essentially of glycerin. In some embodiments, the one or more humectants comprises glycerin in a concentration of about 1.9% w/w of the composition or formulation. In some embodiments, the one or more humectants comprises glycerin in a concentration of about 2.49% w/w of the composition or formulation. In some embodiments, the one or more humectants comprises glycerin in a concentration of about 2.991% w/w of the composition or formulation. In some embodiments, the two or more, three or more, four or more, five or more, six or more, seven or more, eight or more, or nine or more humectants comprises glycerin in a concentration of about 1.9% w/w, about 2.49% w/w, or about 2.991% w/w of the composition or formulation. In some embodiments, the one or more humectants comprises glycerin in a concentration of about 2.991% w/w of the composition or formulation mixed with aqua as a solvent at a concentration of about 0.009% w/w of the composition or formulation. In some embodiments, the humectant and aqua mixed together are added to the formulation in concentration of about 2.991% w/w and about 0.009% w/w respectively. In some embodiments, the mixture of humectant and aqua total about 3.000% w/w of the composition or formulation. In some embodiments, the composition or formulation comprises about 1.9% w/w of glycerin. In some embodiments, the composition or formulation comprises about 2.49% w/w of glycerin. In some embodiments, the composition or formulation comprises about 2.991% w/w of glycerin. In some embodiments, the composition or formulation comprises about 2.991% w/w of glycerin. In some embodiments, the composition or formulation comprises about 3.0% w/w of glycerin. In some embodiments, the composition or formulation comprises about 3.01% w/w of glycerin. In some embodiments, the one or more humectants comprises ethylhexylglycerin. In some embodiments, the two or more, three or more, four or more, five or more, six or more, seven or more, eight or more, or nine or more humectants comprises ethylhexylglycerin. In some embodiments, the composition or formulation comprises about 0.05% w/w of ethylhexylglycerin. In some embodiments, the composition or formulation comprises about 0.075% w/w of ethylhexylglycerin. In some embodiments, the composition or formulation comprises about 0.1% w/w of ethylhexylglycerin. In some embodiments, the composition or formulation comprises about 0.125% w/w of ethylhexylglycerin. In some embodiments, the one or more humectants comprises sodium hyaluronate. In some embodiments, the two or more, three or more, four or more, five or more, six or more, seven or more, eight or more, or nine or more humectants comprises sodium hyaluronate. In some embodiments, the composition or formulation comprises about 0.001, 0.002, 0.003, 0.004, 0.005, 0.006, 0.007, 0.008, 0.009, 0.01, 0.11, 0.12, 0.13, 0.14, 0.015, 0.02, 0.025, 0.03, 0.035, 0.04, 0.05, 0.06, 0.07, 0.08, 0.09, 0.10, 0.15, 0.20, or 0.25% w/w of sodium hyaluronate. In some embodiments, the one or more humectants comprises hyaluronic acid. In some embodiments, the two or more, three or more, four or more, five or more, six or more, seven or more, eight or more, or nine or more humectants comprises hyaluronic acid. In some embodiments, the composition or formulation comprises about 0.001, 0.002, 0.003, 0.004, 0.005, 0.006, 0.007, 0.008, 0.009, 0.01, 0.11, 0.12, 0.13, 0.14, 0.015, 0.02, 0.025, 0.03, 0.035, 0.04, 0.05, 0.06, 0.07, 0.08, 0.09, 0.10, 0.15, 0.20, or 0.25% w/w of hyaluronic acid. In some embodiments, the one or more humectants comprises hydrolyzed hyaluronic acid. In some embodiments, the two or more, three or more, four or more, five or more, six or more, seven or more, eight or more, or nine or more humectants comprises hydrolyzed hyaluronic acid. In some embodiments, the composition or formulation comprises about 0.001, 0.002, 0.003, 0.004, 0.005, 0.006, 0.007, 0.008, 0.009, 0.01, 0.11, 0.12, 0.13, 0.14, 0.015, 0.02, 0.025, 0.03, 0.035, 0.04, 0.05, 0.06, 0.07, 0.08, 0.09, 0.10, 0.15, 0.20, or 0.25% w/w of hydrolyzed hyaluronic acid. In some embodiments, the one or more humectants comprises acetylated sodium hyaluronate. In some embodiments, the two or more, three or more, four or more, five or more, six or more, seven or more, eight or more, or nine or more humectants comprises acetylated sodium hyaluronate. In some embodiments, the composition or formulation comprises about 0.001, 0.002, 0.003, 0.004, 0.005, 0.006, 0.007, 0.008, 0.009, 0.01, 0.11, 0.12, 0.13, 0.14, 0.015, 0.02, 0.025, 0.03, 0.035, 0.04, 0.05, 0.06, 0.07, 0.08, 0.09, 0.10, 0.15, 0.20, or 0.25% w/w of acetylated sodium hyaluronate. In some embodiments, the one or more humectants comprises sodium hyaluronate crosspolymer. In some embodiments, the two or more, three or more, four or more, five or more, six or more, seven or more, eight or more, or nine or more humectants comprises sodium hyaluronate crosspolymer. In some embodiments, the composition or formulation comprises about 0.001, 0.002, 0.003, 0.004, 0.005, 0.006, 0.007, 0.008, 0.009, 0.01, 0.11, 0.12, 0.13, 0.14, 0.015, 0.02, 0.025, 0.03, 0.035, 0.04, 0.05, 0.06, 0.07, 0.08, 0.09, 0.10, 0.15, 0.20, or 0.25% w/w of sodium hyaluronate crosspolymer. In some embodiments, the composition or formulation comprises two or more humectants selected from butylene glycol, cyclomethicone, dipropylene ethylhexylglycerin, glycol, glycerin, propylene glycol, sodium lactate, sorbitol, trehalose, xylitol, or sorbitol, or any combination thereof. In some embodiments, the composition or formulation comprises about 0.01% w/w of sodium hyaluronate. In some embodiments, the composition or formulation comprises about 0.05% w/w of sodium hyaluronate. In some embodiments, the composition or formulation comprises about 0.1% w/w of sodium hyaluronate. In some embodiments, the composition or formulation comprises about 0.01% w/w of hyaluronic acid. In some embodiments, the composition or formulation comprises about 0.05% w/w of hyaluronic acid. In some embodiments, the composition or formulation comprises about 0.1% w/w of hyaluronic acid. In some embodiments, the composition or formulation comprises about 0.01% w/w of hydrolyzed hyaluronic acid. In some embodiments, the composition or formulation comprises about 0.05% w/w of hydrolyzed hyaluronic acid. In some embodiments, the composition or formulation comprises about 0.1% w/w of hydrolyzed hyaluronic acid. In some embodiments, the composition or formulation comprises about 0.01% w/w of acetylated sodium hyaluronate. In some embodiments, the composition or formulation comprises about 0.05% w/w of acetylated sodium hyaluronate. In some embodiments, the composition or formulation comprises about 0.1% w/w of acetylated sodium hyaluronate. In some embodiments, the composition or formulation comprises about 0.01% w/w of sodium hyaluronate crosspolymer. In some embodiments, the composition or formulation comprises about 0.05% w/w of sodium hyaluronate crosspolymer. In some embodiments, the composition or formulation comprises about 0.1% w/w of sodium hyaluronate crosspolymer. In some embodiments, the one or more humectants comprises glycerin, ethylhexylglycerin, and sodium hyaluronate. In some embodiments, the two or more, three or more, four or more, five or more, six or more, seven or more, eight or more, or nine or more humectants comprises glycerin, ethylhexylglycerin, and sodium hyaluronate. In some embodiments, the one or more humectants comprises glycerin at about 2.991% w/w, ethylhexylglycerin at about 0.1% w/w, and sodium hyaluronate at about 0.01% w/w. In some embodiments, the two or more, three or more, four or more, five or more, six or more, seven or more, eight or more, or nine or more humectants comprises glycerin at about 2.991% w/w, ethylhexylglycerin at about 0.1% w/w, and sodium hyaluronate at about 0.01% w/w. In some embodiments, the one or more humectants comprises glycerin, ethylhexylglycerin, and hyaluronic acid. In some embodiments, the two or more, three or more, four or more, five or more, six or more, seven or more, eight or more, or nine or more humectants comprises glycerin, ethylhexylglycerin, and hyaluronic acid. In some embodiments, the one or more humectants comprises glycerin at about 2.991% w/w, ethylhexylglycerin at about 0.075% w/w, and hyaluronic acid at about 0.01% w/w. In some embodiments, the two or more, three or more, four or more, five or more, six or more, seven or more, eight or more, or nine or more humectants comprises glycerin at about 2.991% w/w, ethylhexylglycerin at about 0.075% w/w, and hyaluronic acid at about 0.01% w/w. In some embodiments, the one or more humectants comprises glycerin, ethylhexylglycerin, and hydrolyzed hyaluronic acid. In some embodiments, the two or more, three or more, four or more, five or more, six or more, seven or more, eight or more, or nine or more humectants comprises glycerin, ethylhexylglycerin, and hydrolyzed hyaluronic acid. In some embodiments, the one or more humectants comprises glycerin at about 3.0% w/w, ethylhexylglycerin at about 0.075% w/w, and hydrolyzed hyaluronic acid at about 0.1% w/w. In some embodiments, the two or more, three or more, four or more, five or more, six or more, seven or more, eight or more, or nine or more humectants comprises glycerin at about 3.0% w/w, ethylhexylglycerin at about 0.075% w/w, and hydrolyzed hyaluronic acid at about 0.1% w/w. In some embodiments, the one or more humectants comprises glycerin, ethylhexylglycerin, and acetylated sodium hyaluronate. In some embodiments, the two or more, three or more, four or more, five or more, six or more, seven or more, eight or more, or nine or more humectants comprises glycerin, ethylhexylglycerin, and acetylated sodium hyaluronate. In some embodiments, the one or more humectants comprises glycerin at about 2.991% w/w, ethylhexylglycerin at about 0.1% w/w, and acetylated sodium hyaluronate at about 0.05% w/w. In some embodiments, the two or more, three or more, four or more, five or more, six or more, seven or more, eight or more, or nine or more humectants comprises glycerin at about 2.991% w/w, ethylhexylglycerin at about 0.1% w/w, and acetylated sodium hyaluronate at about 0.05% w/w. In some embodiments, the one or more humectants comprises glycerin, ethylhexylglycerin, and sodium hyaluronate crosspolymer. In some embodiments, the two or more, three or more, four or more, five or more, six or more, seven or more, eight or more, or nine or more humectants comprises glycerin, ethylhexylglycerin, and sodium hyaluronate crosspolymer. In some embodiments, the one or more humectants comprises glycerin at about 2.991% w/w, ethylhexylglycerin at about 0.05% w/w, and sodium hyaluronate crosspolymer at about 0.01% w/w. In some embodiments, the two or more, three or more, four or more, five or more, six or more, seven or more, eight or more, or nine or more humectants comprises glycerin at about 2.991% w/w, ethylhexylglycerin at about 0.05% w/w, and sodium hyaluronate crosspolymer at about 0.01% w/w.

### Preservatives and Antioxidants

A pharmaceutically acceptable preservative can be employed to increase the shelf life of the composition or formulation. In some embodiments of compositions or formulations described herein, one or more preservatives are present. In some embodiments of compositions or formulations described herein, two or more, three or more, four or more, five or more, six or more, seven or more, eight or more, or nine or more preservatives or antioxidants are present. Suitable preservatives or antioxidants for use in compositions or formulations described here include phenoxyethanol, ethylhexylglycerin, benzalkonium chloride, benzyl alcohol, phenol, urea, parabens, butylated hydroxytoluene (BHT), butylated hydroxyanisole (BHA), tocopherol, or the like, and mixtures thereof, can be employed. Phenoxyethanol is a colorless, oily liquid that's used as a preservative. Phenoxyethanol is effective against many types of bacteria and yeasts. Ethylhexylglycerin can function as a preservative, a humectant, or any combination thereof in compositions or formulations described herein. Ethylhexylglycerin helps prevent bacteria growth. Phenoxyethanol and ethylhexylglycerin can be produced synthetically. In some embodiments of compositions or formulations described herein, the one or more preservatives comprise at least about 0.1, 0.2, 0.3, 0.4, 0.5, 0.6, 0.7, 0.8, 0.9, 1.0, or 1.1, or 1.25% w/w of the composition or formulation. In some embodiments of compositions or formulations described herein, the two or more, three or more, four or more, five or more, six or more, seven or more, eight or more, or nine or more preservatives or antioxidants comprise at least about 0.1, 0.2, 0.3, 0.4, 0.5, 0.6, 0.7, 0.8, 0.9, 1.0, or 1.1, or 1.25% w/w of the composition or formulation. In some embodiments of compositions or formulations described herein, the one or more preservatives comprise less than about 5.0, 4.0, 3.0, 2.0, 1.5, 1.25, or 1.1% w/w of the composition or formulation. In some embodiments of compositions or formulations described herein, the two or more, three or more, four or more, five or more, six or more, seven or more, eight or more, or nine or more preservatives or antioxidants comprise less than about 5.0, 4.0, 3.0, 2.0, 1.5, 1.25, or 1.1% w/w of the composition or formulation. In some embodiments of compositions or formulations described herein, the one or more preservatives comprise about 1.0% w/w of the composition or formulation. In some embodiments of compositions or formulations described herein, the two or more, three or more, four or more, five or more, six or more, seven or more, eight or more, or nine or more preservatives or antioxidants comprise about 1.0% w/w of the composition or formulation. In some embodiments, the composition or formulation comprises about 0.1, 0.2, 0.3, 0.4, 0.5, 0.6, 0.7, 0.8, 0.9, or 1.0% w/w of phenoxyethanol. In some embodiments, the composition or formulation comprises about 0.7% w/w of phenoxyethanol. In some embodiments, the composition or formulation comprises about 0.8% w/w of phenoxyethanol. In some embodiments, the composition or formulation comprises about 0.9% w/w of phenoxyethanol. In some embodiments, the composition or formulation comprises about 1.0% w/w of phenoxyethanol. In some embodiments, the composition or formulation comprises about 0.1, 0.2, 0.3, 0.4, 0.5, 0.6, 0.7, 0.8, 0.9, or 1.0% w/w of ethylhexylglycerin. In some embodiments, the composition or formulation comprises about 0.1% w/w of ethylhexylglycerin. In some embodiments, the composition or formulation comprises about 0.2% w/w of ethylhexylglycerin. In some embodiments, the composition or formulation comprises about 0.3% w/w of ethylhexylglycerin. In some embodiments, the composition or formulation comprises about 0.9% w/w of phenoxyethanol and about 0.1% w/w of ethylhexylglycerin. In some embodiments, the composition or formulation comprises about 0.8% w/w of phenoxyethanol and about 0.2% w/w of ethylhexylglycerin. In some embodiments, the composition or formulation comprises about 0.7% w/w of phenoxyethanol and about 0.3% w/w of ethylhexylglycerin. In some embodiments, the one or more preservatives present in the composition or formulation consist essentially of phenoxyethanol and ethylhexylglycerin. In some embodiments, the two or more preservatives or antioxidants present in the composition or formulation consist essentially of phenoxyethanol and ethylhexylglycerin. In some embodiments, the one or more preservatives present in the composition or formulation consist essentially of phenoxyethanol at about 0.9% w/w and ethylhexylglycerin at about 0.1% w/w. In some embodiments, the two or more preservatives or antioxidants present in the composition or formulation consist essentially of phenoxyethanol at about 0.9% w/w and ethylhexylglycerin at about 0.1% w/w. In some embodiments, the one or more preservatives present in the composition or formulation consist essentially of phenoxyethanol at about 0.8% w/w and ethylhexylglycerin at about 0.2% w/w. In some embodiments, the two or more preservatives or antioxidants present in the composition or formulation consist essentially of phenoxyethanol at about 0.8% w/w and ethylhexylglycerin at about 0.2% w/w. In some embodiments, the one or more preservatives present in the composition or formulation consist essentially of phenoxyethanol at about 0.7% w/w and ethylhexylglycerin at about 0.3% w/w. In some embodiments, the two or more preservatives or antioxidants present in the composition or formulation consist essentially of phenoxyethanol at about 0.7% w/w and ethylhexylglycerin at about 0.3% w/w.

### Buffers

A buffering agent can be employed to stabilize the pH of the composition or formulation. One effect of stabilized pH is increased shelf life of the composition or formulation. In some embodiments of compositions or formulations described herein, one or more buffering agents are present. In some embodiments, the one or more buffering agents comprise sodium hydroxide, hydrochloric acid, sodium phosphate monobasic, sodium phosphate dibasic, or sodium citrate, or a combination thereof. In some embodiments, the one or more buffering agents is dissolved in a solvent. In some embodiments, the one or more buffering agents is dissolved in aqua. In some embodiments, the one or more buffering agents serves to adjust the pH of the composition or formulation. In some embodiments, the one or more buffering agents comprises sodium hydroxide. In some embodiments of compositions or formulations described herein, two or more, three or more, four or more, or five or more buffering agents are present. In some embodiments, the two or more, three or more, four or more, or five or more buffering agents comprise sodium hydroxide, hydrochloric acid, sodium phosphate monobasic, sodium phosphate dibasic, or sodium citrate, or a combination thereof. In some embodiments, the composition or formulation comprises sodium hydroxide at a concentration of about 0.005, 0.0075, 0.01, 0.0125, 0.015, 0.0175, 0.02, 0.025 0.05, 0.07, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30% w/w. In some embodiments, the composition or formulation comprises sodium hydroxide at a concentration of about 0.1% w/w. In some embodiments, the composition or formulation comprises sodium hydroxide at a concentration of about 0.12% w/w. In some embodiments, the composition or formulation comprises sodium hydroxide at a concentration of about 0.14% w/w. In some embodiments, the composition or formulation comprises sodium hydroxide at a concentration of about 0.16% w/w. In some embodiments, the composition or formulation comprises sodium hydroxide at a concentration of about 0.18% w/w. In some embodiments, the composition or formulation comprises sodium hydroxide at a concentration of about 0.2% w/w. In some embodiments, the composition or formulation comprises sodium hydroxide at a concentration of about 0.10% w/w dissolved in about 0.70% w/w of aqua. In some embodiments, the composition or formulation comprises sodium hydroxide at a concentration of about 0.12% w/w dissolved in about 0.68% w/w of aqua. In some embodiments, the composition or formulation comprises sodium hydroxide at a concentration of about 0.14% w/w dissolved in about 0.66% w/w of aqua. In some embodiments, the composition or formulation comprises sodium hydroxide at a concentration of about 0.16% w/w dissolved in about 0.64% w/w of aqua. In some embodiments, the composition or formulation comprises sodium hydroxide at a concentration of about 0.18% w/w dissolved in about 0.62% w/w of aqua. In some embodiments, the composition or formulation comprises sodium hydroxide at a concentration of about 0.20% w/w dissolved in about 0.60% w/w of aqua. In some embodiments, the composition or formulation comprises sodium hydroxide at a concentration of about 0.10% w/w dissolved in about 0.70% w/w of aqua that is added to the composition or formulation to adjust the pH. In some embodiments, the composition or formulation comprises sodium hydroxide at a concentration of about 0.16% w/w dissolved in about 0.64% w/w of aqua that is added to the composition or formulation to adjust the pH. In some embodiments, the composition or formulation comprises sodium hydroxide at a concentration of about 0.10% w/w dissolved in about 0.70% w/w of aqua that is added to the composition or formulation to stabilize the pH. In some embodiments, the composition or formulation comprises sodium hydroxide at a concentration of about 0.16% w/w dissolved in about 0.64% w/w of aqua that is added to the composition or formulation to stabilize the pH. In some embodiments, the composition or formulation has a pH of from about 4.0 to about 7.0. In some embodiments, the composition or formulation has a pH of about 4.0, about 4.5, about 5.0, about 5.5, about 6.0, about 6.5, or about 7.0. In some embodiments, the composition or formulation has a pH of about 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.6, or 6.8. In some embodiments, one or more bases are added to the composition or formulation to form a buffering system together with one or more acidic components of the composition or formulation.

### Rheology Modifiers

Rheology is the study of how matter deforms and flows, especially fluids, soft solids, and complex materials that deform plastically. Rheology describes the flow dynamics of liquid solutions and the deformation of solids. An aim of rheology studies is to understand, model, and predict behavior of complex materials under conditions of storage and use. The rheology of a particular formulation influences consistency of preparation for each batch produced and affects the look, feel, and consistency of the formulation under various conditions. The particular rheological properties of heterogeneous dispersions, such as micronized benzoyl peroxide formulations described herein, are complex and can be interpreted through several parameters. Rheology studies comprise features such as: 1) flow: how materials move, especially fluid; 2) deformation: how materials change shape when a force is applied; 3) strain: how much a material deforms; 4) strain rate: how quickly a material deforms, and 5) stress: the force applied to a material per unit area. These features may be optimized by selecting particular rheology modifiers, at certain concentrations, for inclusion in the formulation. These features determine the consistency of topical products like creams, gels, gel-creams, lotions, solutions, emulsions, suspensions, or serums, as well as drive decisions in creating formulations having a desired texture and spreadability. A focus on rheology can also help predict how materials will behave in certain conditions, like when pumped, squeezed, or spread. Within the industries such as the cosmetology industry or the pharmaceutical industry, rheology is an important consideration when manufacturing and engineering solutions for topical application, especially gels and non-Newtonian fluids. A surfactant of a formulation is an ingredient used to control surface tension. A rheology modifier of a formulation is an ingredient used to determine or control viscosity of the fluid or different solids. A rheology modifier of a formulation is also an ingredient used to determine or control the rheology profile and delivery of a fluid, a lotion, or the like, which includes yield stress and viscosity at different shear modes and rates. The rheology profile describes the state, spatial arrangement, and physical attributes of the formulation or of ingredients that are starting materials for the formulation. In a Newtonian liquid, the viscosity is independent of the shear rate, making the liquid ideally viscous. When the viscosity of a fluid changes based on the shear rate, the fluid is classified as a non-Newtonian fluid. Topical creams are considered non-Newtonian, shear-thinning materials, meaning their viscosity is high at low shear rates and low at high ones. A topical formulation is generally considered non-Newtonian rather than Newtonian in conditions of use, meaning that its viscosity changes depending on the applied shear rate, which is important for achieving good spreadability on the skin when applied. Most creams and lotions exhibit this behavior, making them easier to apply with increasing shear stress. The non-Newtonian property of topical formulations, topical creams, and the like, is desirable for topical products as this enables smooth application and even distribution on the skin surface of a subject.

Analysis of the rheological behavior of formulations may be characterized by various methods, such as using a rheometer that is appropriate for monitoring the non-Newtonian flow behavior of the formulation. Types of rheometers that may be used in this analysis include: a capillary rheometer, a rotational rheometer, a dynamic shear stress rheometer, a cone-plate rheometer, and a falling ball rheometer. While a rheometer measures how a fluid flows and its viscoelastic properties, a viscometer measures viscosity of a fluid. A viscometer may also be used to measure viscosity of a formulation using or one or more types of viscometers such as capillary, falling ball, cone-plate, falling piston, falling sphere, rolling ball, viscosity cup, digital, and orifice viscometers. A complete flow curve analysis of shear stress (or viscosity) versus shear rate may be indicated to determine the characteristics and limits of non-Newtonian flow behavior of a formulation under various conditions. A complete flow curve of shear stress versus shear rate comprises multiple data points across a range of attainable shear rates, until low or high shear plateaus are identified. Through this analysis, the linear viscoelastic response (storage and loss modulus versus frequency) is measured and recorded. This analysis requires measuring the viscosity of the test formulation over a wide variety of shear rates. The Newtonian plateau portions of the flow curve with a zero-shear viscosity are identified at one end of the plotted curve and an infinite-shear viscosity is identified at the other end of the plot. The shear thinning portion (non-Newtonian) of the curve resides in between these two plateaus, and the rheology modifiers of the formulation may be optimized by selecting particular rheology modifiers at certain concentrations, to produce a favorable extent of non-Newtonian flow behavior. Shear stress is a measure of how hard a material is pushed. Shear rate is a measure of how fast a material flows. Viscosity is the determined ratio of shear stress over shear rate. Suspensions, emulsions and gels, among other liquid composition formats, all exhibit non-Newtonian shear thinning behavior, meaning that their viscosity is dependent on shear rate and that the viscosity decreases at higher shear rates. Yield stress is another key contribution to the rheology of a formulation and contributes to application properties, sensory properties, and delivery of the formulation from a container. Yield stress of the formulation may be optimized by selecting particular rheology modifiers at certain concentrations. Yield stress is defined as the strength of a soft solid structure resulting from interactions of particles, droplets, polymers, and surfactants. Yield stress can be measured in a number of different ways, one of which is through oscillatory stress sweeps. In oscillatory stress sweep tests, the oscillatory testing measures phase angle (in°) against stress (in Pascals, or Pa). The phase angle is effectively a measure of structure. With a phase angle of 0°, the structure of the formulation is intact. With a phase angle of 90°, the structure of the formulation is disrupted. Phase angle changes as stress is increased. An increase in phase angle indicates a destruction of structure as the yield proceeds, and it is at that point that the yield stress of the sample has been identified. Linear viscoelastic response is another rheological feature that may be tested and optimized in formulations. A linear viscoelastic response examines storage modulus and loss of modulus as a function of frequency. Storage modulus (G′), describes solid-like contributions (elastic). Loss modulus (G″) describes liquid-like contributions (viscous). G′ and G″ both contribute to Complex modulus (G*) which is a metric of overall rigidity. In a linear viscoelastic response test, a sample is oscillated at a range of frequencies effectively imposing a range of deformation time scales on the sample. By examining the relative relationship between the moduli across the range of frequencies, features of the microstructure of the formulation are quantified. A topical cream with a desirable rheological profile exhibits an elastic dominant response, with Storage modulus (G′) being higher than Loss modulus (G″) across the range of frequencies. For a well-formed elastic gel structure typical of an emulsion, the Storage modulus dominates Loss modulus throughout the range of frequencies tested (measured by angular frequency in rad/s). For the relaxable, weak interactions typical of a polymer or surfactant solution, Storage modulus is unable to dominate Loss modulus at lower frequencies tested. Creep testing is another way to examine the rheology of a formulation and may be used to optimize concentrations of ingredients in the formulation. Creep testing involves applying a small stress to a sample, maintaining that stress over a period of time, and monitoring the strain (as an amount of deformation) over time. During a creep test, a test sample undergoes an initially, highly elastic deformation, but eventually, the deformation rate slows to a steady state viscous flow and the plot straightens. The slope in the steady state provides a calculated shear rate. By dividing the applied stress by the shear rate, a viscosity is determined. As this viscosity is obtained at very low stresses, this viscosity is indicative of the viscosity at a close-to-rest condition. A Pascal-second (symbol: Pa·s)\ is the SI unit of viscosity, equivalent to newton-second per square meter (N·s m²). It is sometimes referred to as the “poiseuille” (PI). One poiseuille is exactly 0.1 Pa·s. One poiseuille is 10 poise or 1000 centipoises (cP), while 1 cP=1 mPa·s (one millipascal-second).

Viscosity measurements are used to check the quality of materials used in the formulation and to ensure consistent texture. Formulations described herein are formulated to have a viscosity that falls within a ideal range for the type of delivery. In some embodiments, a topical cream formulation comprising micronized BPO has a viscosity within the range of about 2,000 centipoises (cP) to about 50,000 cP. This ensures good spreadability of the formulation while still maintaining enough consistency to adhere to the skin and deliver the micronized BPO effectively. In some embodiments, a topical gel formulation comprising micronized BPO has a viscosity within the range of about 2,000 cP to about 4,000 cP. In some embodiments, a topical gel-cream formulation comprising micronized BPO has a viscosity within the range of about 35,000 cP to about 40,000 cP. In some embodiments, a topical lotion formulation comprising micronized BPO has a viscosity within the range of about 2,000 cP to about 50,000 cP. In some embodiments, a topical emulsion formulation comprising micronized BPO has a viscosity within the range of about 1,000 cP to about 10,000 cP. In some embodiments, a topical suspension emulsion formulation comprising micronized BPO has a viscosity within the range of about 1,000 cP to about 5,000 cP. In some embodiments, a topical serum formulation comprising micronized BPO has a viscosity within the range of about 200 cP to about 1,000 cP. In some embodiments, a topical formulation comprising micronized BPO comprises one or more rheology modifiers in a sufficient amount to achieve a desired viscosity when measured at room temperature. In some embodiments, the one or more rheology modifiers are included in a sufficient quantity to produce a viscosity measurement in the formulation of at least 200 cP, 300 cP, 400 cP, 500 cP, 600 cP, 700 cP, 800 cP, 900 cP, 1000 cP, 1100 cP, 1200 cP, 1300 cP, 1400 cP, 1500 cP, 1600 cP, 1700 cP, 1800 cP, 1900 cP, 2000 cP, 2250 cP, 2500 cP, 3000 cP, 3500 cP, 4000 cP, 4500 cP, 5000 cP, 5500 cP, 6000 cP, 6500 cP, 7000 cP, 7500 cP, 7500 cP, 8000 cP, 8500 cP, 9000 cP, 9500 cP, 10000 cP, 11000 cP, 12000 cP, 13000 cP, 14000 cP, 15000 cP, 16000 cP, 17000 cP, 18000 cP, 19000 cP, 20000 cP, 22000 cP, 24000 cP, 26000 cP, 28000 cP, 30000 cP, 32500 cP, 35000 cP, 37500 cP, 40000 cP, 42500 cP, 45000 cP, 47500 cP, or 50,000 cP, or more than 50,000 cP when measured at about room temperature and at about 1 atmosphere of pressure. In some embodiments, the one or more rheology modifiers are included in a sufficient quantity to produce a viscosity measurement in the formulation of less than 200 cP, 300 cP, 400 cP, 500 cP, 600 cP, 700 cP, 800 cP, 900 cP, 1000 cP, 1100 cP, 1200 cP, 1300 cP, 1400 cP, 1500 cP, 1600 cP, 1700 cP, 1800 cP, 1900 cP, 2000 cP, 2250 cP, 2500 cP, 3000 cP, 3500 cP, 4000 cP, 4500 cP, 5000 cP, 5500 cP, 6000 cP, 6500 cP, 7000 cP, 7500 cP, 7500 cP, 8000 cP, 8500 cP, 9000 cP, 9500 cP, 10000 cP, 11000 cP, 12000 cP, 13000 cP, 14000 cP, 15000 cP, 16000 cP, 17000 cP, 18000 cP, 19000 cP, 20000 cP, 22000 cP, 24000 cP, 26000 cP, 28000 cP, 30000 cP, 32500 cP, 35000 cP, 37500 cP, 40000 cP, 42500 cP, 45000 cP, 47500 cP, or 50,000 cP, when measured at about room temperature and at about 1 atmosphere of pressure. In some embodiments, the one or more rheology modifiers are included in a sufficient quantity to produce a viscosity measurement in the formulation of about 100 cP, about 200 cP, about 300 cP, about 350 cP, about 400 cP, about 500 cP, about 6000 cP, about 750 cP, about 850 cP, about 1000 cP, about 1500 cP, about 2000 cP, about 3000 cP, about 4000 cP, about 5000 cP, about 6000 cP, about 7000 cP, about 8000 cP, about 9000 cP, about 10,000 cP, about 12500 cP, about 15000 cP, about 17500 cP, about 20000 cP, about 22500 cP, about 25000 cP, about 27500 cP, about 30000 cP, about 35000 cP, about 40000 cP, about 45000 cP, or about 50000 cP.

The basic rheology-determining parameters of an emulsion, a lotion, or similar formulation of liquid solution described herein are considered a continuous phase rheology and determine the nature of the drops (e.g., size distribution, deformability, internal viscosity, concentration of particles and particles interactions, etc.). The rheology modifiers and concentrations thereof were selected to create a rheology profile for precise delivery of the micronized BPO. The rheology profile for the formulation includes the yield stress and the viscosity at different shear modes and rates. The rheology profile of the formulation provides a functional formulation network, provides an easy to apply structure in viscosity, and generates favorable glide and suspension properties. In formulations comprising micronized BPO, preventing settling of the micronized BPO is a key concern for ensuring consistency during use of the product and to prevent settling in packaged product. When a formulation has solid pigments or particles like micronized BPO, rheology modifiers help to suspend these particles evenly throughout the product to prevent any settling or sedimentation and help with uniform distribution and effective delivery. Rheology modifiers help to control viscosity and flow, to improve consistency of application and extend useable lifetimes of these micronized BPO systems and packaged formulations. Besides just viscosity, the structure and solid-like properties of micro-dispersions play a crucial role in dispersion attributes and behaviors of topical formulations comprising micronized BPO. In formulations described herein, one or more rheology modifiers contribute to rheology of the formulation system that holds active ingredient (e.g., micronized BPO) and helps with suspending, delivery, and uniform distribution upon topical application of the formulation. The rheology of a topical formulation impacts with micronized benzoyl peroxide by significantly improving its ability to spread and penetrate the skin due to its smaller micronized particle size, allowing for better distribution of the BPO within the formulated product, potentially leading to increased efficacy and reduced irritation compared to standard benzoyl peroxide particles; essentially making the formulated product smoother and easier to apply while enhancing its penetration into the skin's pores. By distributing the active ingredient of micronized benzoyl peroxide more evenly, rheology modifiers can minimize irritation caused by concentrated clusters of large particles. In some embodiments of formulations described herein, the one or more rheology modifiers comprise an agar, an alginate, an ammonium acryloyldimethyltaurate/VP copolymer, a pectin, an acacia, a tragacanth, a Karaya gum, a guar gum, a xanthan gum, a starch, a cellulose, a carbomer, a beeswax, a candelilla wax, or a carnauba wax, or any combination thereof. In some embodiments of formulations described herein, two or more rheology modifiers contribute to rheology of the formulation system that holds active ingredient (e.g., micronized BPO) and help with suspending, delivery, and uniform distribution upon topical application of the formulation. In some embodiments, the two or more rheology modifiers are selected from an agar, an alginate, an ammonium acryloyldimethyltaurate/VP copolymer, a pectin, an acacia, a tragacanth, a Karaya gum, a guar gum, a xanthan gum, a starch, a cellulose, a carbomer, a beeswax, a candelilla wax, or a carnauba wax, or any combination thereof. In some embodiments of formulations described herein, three or more rheology modifiers contribute to rheology of the formulation system that holds active ingredient (e.g., micronized BPO) and help with suspending, delivery, and uniform distribution upon topical application of the formulation. In some embodiments, the three or more rheology modifiers are selected from an agar, an alginate, an ammonium acryloyldimethyltaurate/VP copolymer, a pectin, an acacia, a tragacanth, a Karaya gum, a guar gum, a xanthan gum, a starch, a cellulose, a carbomer, a beeswax, a candelilla wax, or a carnauba wax, or any combination thereof. In some embodiments of formulations described herein, xanthan gum is selected for inclusion in the formulation as a rheology modifier. Xanthan gum is a rheology control agent (rheology modifier) that improves the texture, stability, and appearance of the formulation. Xanthan gum is a naturally occurring polysaccharide that is nontoxic and non-irritating. Inclusion of a selected amount of xanthan gum in the formulation makes the product feel smoother and silkier upon application to the face of the subject and can help reduce sensory irritation by forming a protective barrier on the skin. Because of its excellent viscosity properties, thixotropy or pseudo-plasticity, xanthan gum is used to adjust the cosmetics rheological properties and improve the cosmetics touch after use for formulations described herein. In some embodiments, xanthan gum is included in the formulation at a concentration of about 0.01% w/w, about 0.02% w/w, about 0.03% w/w, about 0.04% w/w, about 0.05% w/w, about 0.06% w/w, about 0.07% w/w, about 0.08% w/w, about 0.09% w/w, about 0.10% w/w, about 0.11% w/w, about 0.12% w/w, about 0.13% w/w, about 0.14% w/w, about 0.15% w/w, about 0.16% w/w, about 0.17% w/w, about 0.18% w/w, about 0.19% w/w, about 0.20% w/w, about 0.22% w/w, about 0.25% w/w, about 0.27% w/w, about 0.30% w/w, about 0.35% w/w, about 0.40% w/w, about 0.45% w/w, or about 0.50% w/w. In some embodiments, xanthan gum is included in the formulation at a concentration of about 0.05% w/w. In some embodiments, xanthan gum is included in the formulation at a concentration of about 0.10% w/w. In some embodiments, xanthan gum is included in the formulation at a concentration of about 0.15% w/w. In some embodiments, xanthan gum is included in the formulation at a concentration of about 0.20% w/w. In some embodiments, xanthan gum is included in the formulation at a concentration of about 0.25% w/w. In some embodiments, xanthan gum is included in the formulation at a concentration of about 0.30% w/w. In some embodiments of formulations described herein, carbomer is selected for inclusion in the formulation as a rheology modifier. Carbomer primarily functions as a rheology modifier, meaning it controls the consistency and flow of a formulated product by significantly increasing its viscosity, essentially acting as a thickener to create gel form with a smooth, spreadable texture while maintaining stability and preventing separation of ingredients of the formulation. This allows for even application and improved delivery of active ingredients (e.g., micronized BPO) to the skin of the subject. By controlling viscosity, carbomer contributes to a desirable texture and feel on the skin following topical application, making products easier to spread and apply. By controlling viscosity, carbomer contributes to a desirable texture and feel on the skin and creates an easy like water break sensation that helps transferring active ingredient. In some embodiments, carbomer is included in the formulation at a concentration of about 0.05% w/w, about 0.10% w/w, about 0.15% w/w, about 0.20% w/w, about 0.25% w/w, about 0.275% w/w, about 0.30% w/w, about 0.325% w/w, about 0.35% w/w, about 0.375% w/w, about 0.40% w/w, about 0.425% w/w, about 0.45% w/w, about 0.475% w/w, about 0.5% w/w, about 0.525% w/w, about 0.55% w/w, about 0.575% w/w, about 0.60% w/w, about 0.65% w/w, about 0.70% w/w, about 0.75% w/w, about 0.80% w/w, about 0.85% w/w, about 0.90% w/w, about 0.95% w/w, or about 1.00% w/w. In some embodiments, carbomer is included in the formulation at a concentration of about 0.25% w/w. In some embodiments, carbomer is included in the formulation at a concentration of about 0.30% w/w. In some embodiments, carbomer is included in the formulation at a concentration of about 0.35% w/w. In some embodiments, carbomer is included in the formulation at a concentration of about 0.40% w/w. In some embodiments, carbomer is included in the formulation at a concentration of about 0.45% w/w. In some embodiments, carbomer is included in the formulation at a concentration of about 0.50% w/w. In some embodiments of formulations described herein, ammonium acryloyldimethyltaurate/VP copolymer is selected for inclusion in the formulation as a rheology modifier. In formulations described herein, ammonium acryloyldimethyltaurate/VP copolymer forms a cross-linked polymer network that traps particles of micronized BPO, creating a stable structure and keeping the BPO particles in the form and sizes that are able to penetrate deeper into the acne pores of the subject. Ammonium acryloyldimethyltaurate/VP copolymer also works as thickening and gelling agent plus as texturizer and provides a light, fresh, and non-tacky skin feel. In some embodiments, ammonium acryloyldimethyltaurate/VP copolymer is included in the formulation at a concentration of about 0.05% w/w, about 0.10% w/w, about 0.15% w/w, about 0.20% w/w, about 0.25% w/w, about 0.275% w/w, about 0.30% w/w, about 0.325% w/w, about 0.35% w/w, about 0.375% w/w, about 0.40% w/w, about 0.425% w/w, about 0.45% w/w, about 0.475% w/w, about 0.5% w/w, about 0.525% w/w, about 0.55% w/w, about 0.575% w/w, about 0.60% w/w, about 0.65% w/w, about 0.70% w/w, about 0.75% w/w, about 0.80% w/w, about 0.85% w/w, about 0.90% w/w, about 0.95% w/w, or about 1.00% w/w. In some embodiments, ammonium acryloyldimethyltaurate/VP copolymer is included in the formulation at a concentration of about 0.25% w/w. In some embodiments, ammonium acryloyldimethyltaurate/VP copolymer is included in the formulation at a concentration of about 0.30% w/w. In some embodiments, ammonium acryloyldimethyltaurate/VP copolymer is included in the formulation at a concentration of about 0.35% w/w. In some embodiments, ammonium acryloyldimethyltaurate/VP copolymer is included in the formulation at a concentration of about 0.40% w/w. In some embodiments, ammonium acryloyldimethyltaurate/VP copolymer is included in the formulation at a concentration of about 0.45% w/w. In some embodiments, ammonium acryloyldimethyltaurate/VP copolymer is included in the formulation at a concentration of about 0.50% w/w. In some embodiments, the formulation comprises three or more rheology modifiers comprising xanthan gum, carbomer, and ammonium acryloyldimethyltaurate/VP copolymer.

### Viscosity Control

A viscosity control agent can be employed to increase or stabilize the viscosity of the composition or formulation. In some embodiments, the viscosity control agent can serve as a thickener for the composition or formulation. In some embodiments of compositions or formulations described herein, one or more viscosity control agents are present. In some embodiments of the compositions or formulations described herein, two or more, three or more, four or more, five or more, six or more, seven or more, eight or more, or nine or more viscosity control agents are present. In some embodiments of compositions or formulations described herein, one or more thickeners are present. In some embodiments of compositions or formulations described herein, two or more, three or more, four or more, five or more, six or more, seven or more, eight or more, or nine or more thickeners are present. In some embodiments, the one or more viscosity control agents comprise agar, alginate, ammonium acryloyldimethyltaurate/vp copolymer, pectin, acacia, tragacanth, Karaya gum, guar gum, xanthan gum, starch, cellulose, carbomer, bees wax, candelilla wax, or carnauba wax, or any combination thereof. In some embodiments, the two or more, three or more, four or more, five or more, six or more, seven or more, eight or more, or nine or more viscosity control agents comprise agar, alginate, ammonium acryloyldimethyltaurate/vp copolymer, pectin, acacia, tragacanth, Karaya gum, guar gum, xanthan gum, starch, cellulose, carbomer, bees wax, candelilla wax, or carnauba wax, or any combination thereof. In some embodiments, the one or more viscosity control agents comprise ammonium acryloyldimethyltaurate/vp copolymer, carbomer, or xanthan gum, or any combination thereof. In some embodiments, the one or more viscosity control agents comprise ammonium acryloyldimethyltaurate/vp copolymer, carbomer, and xanthan gum. In some embodiments, the one or more viscosity control agents consist essentially of ammonium acryloyldimethyltaurate/vp copolymer, carbomer, and xanthan gum. In some embodiments, the three or more viscosity control agents consist essentially of ammonium acryloyldimethyltaurate/vp copolymer, carbomer, and xanthan gum. In some embodiments, the composition comprises ammonium acryloyldimethyltaurate/vp copolymer, carbomer, and xanthan gum. In some embodiments, the formulation comprises ammonium acryloyldimethyltaurate/vp copolymer, carbomer, and xanthan gum. In some embodiments, the ammonium acryloyldimethyltaurate/vp copolymer serves as a gelling agent used in an aqueous solution to form a gel-cream. In some embodiments, the ammonium acryloyldimethyltaurate/vp copolymer serves as a thickener to improve spreading upon topical application. In some embodiments, the ammonium acryloyldimethyltaurate/vp copolymer serves as a stabilizer to stabilize the composition or formulation without a need for additional emulsifiers. In some embodiments, the ammonium acryloyldimethyltaurate/vp copolymer serves as a texturizer to improve the sensory texture of a composition or formulation described herein by decreasing stickiness. In some embodiments, the composition or formulation comprises ammonium acryloyldimethyltaurate/vp copolymer of about 0.1, 0.2, 0.3, 0.4, 0.5, 0.6, 0.7, 0.8, 0.9 or 1.0% w/w. In some embodiments, the composition or formulation comprises ammonium acryloyldimethyltaurate/vp copolymer of about 0.2% w/w. In some embodiments, the composition or formulation comprises ammonium acryloyldimethyltaurate/vp copolymer of about 0.3% w/w. In some embodiments, the composition or formulation comprises ammonium acryloyldimethyltaurate/vp copolymer of about 0.4% w/w. In some embodiments, the composition or formulation comprises ammonium acryloyldimethyltaurate/vp copolymer of about 0.5% w/w. Carbomers are synthetic polymers that may be used in compositions or formulations described herein and may act as thickeners, suspending agents, or emulsion stabilizers, or any combination thereof. In micronized benzoyl peroxide compositions or formulations described herein that comprise one or more carbomers, the final concentration of salts in the composition or formulation may significantly affect the viscosity contribution of the one or more carbomers to the final viscosity of the composition or formulation. In some embodiments, the composition or formulation comprises Carbomer 974P. In some embodiments, the composition or formulation comprises Carbomer 980. In some embodiments, the composition or formulation comprises Carbomer 940. In some embodiments, the composition or formulation comprises Ultrez 30. In some embodiments, the composition or formulation comprises Ultrez 10. In some embodiments, the composition or formulation comprises a carbomer of about 0.1, 0.2, 0.3, 0.4, 0.5, 0.6, 0.7, 0.8, 0.9 or 1.0% w/w. In some embodiments, the composition or formulation comprises a carbomer of about 0.2% w/w. In some embodiments, the composition or formulation comprises a carbomer of about 0.3% w/w. In some embodiments, the composition or formulation comprises a carbomer of about 0.4% w/w. In some embodiments, the composition or formulation comprises a carbomer of about 0.5% w/w. In some embodiments, the composition or formulation comprises a carbomer of about 0.6% w/w. Xanthan gum is a natural thickener which may be derived from plants. In some embodiments, the composition or formulation comprises xanthan gum. In some embodiments, the composition or formulation comprises xanthan gum which may serve as a viscosity control agent. In some embodiments, the composition or formulation comprises xanthan gum which may serve as a thickener. In some embodiments, the composition or formulation comprises xanthan gum of about 0.1, 0.2, 0.3, 0.4, 0.5, 0.6, 0.7, 0.8, 0.9 or 1.0% w/w. In some embodiments, the composition or formulation comprises xanthan gum of about 0.1% w/w. In some embodiments, the composition or formulation comprises xanthan gum of about 0.2% w/w. In some embodiments, the composition or formulation comprises xanthan gum of about 0.3% w/w. In some embodiments, the composition or formulation comprises xanthan gum of about 0.4% w/w. In some embodiments, the composition or formulation comprises xanthan gum of about 0.5% w/w. In some embodiments, the composition or formulation comprises ammonium acryloyldimethyltaurate/vp copolymer of about 0.4% w/w, a carbomer of about 0.5% w/w, and xanthan gum of about 0.3% w/w. In some embodiments, the viscosity control agents of the composition or formulation comprise ammonium acryloyldimethyltaurate/vp copolymer of about 0.4% w/w, a carbomer of about 0.5% w/w, and xanthan gum of about 0.3% w/w. In some embodiments, the viscosity control agents of the composition or formulation consist essentially of ammonium acryloyldimethyltaurate/vp copolymer of about 0.4% w/w, a carbomer of about 0.5% w/w, and xanthan gum of about 0.3% w/w. In some embodiments, the composition or formulation comprises ammonium acryloyldimethyltaurate/vp copolymer of about 0.4% w/w, a carbomer of about 0.4% w/w, and xanthan gum of about 0.2% w/w. In some embodiments, the viscosity control agents of the composition or formulation comprise ammonium acryloyldimethyltaurate/vp copolymer of about 0.4% w/w, a carbomer of about 0.4% w/w, and xanthan gum of about 0.2% w/w. In some embodiments, the viscosity control agents of the composition or formulation consist essentially of ammonium acryloyldimethyltaurate/vp copolymer of about 0.4% w/w, a carbomer of about 0.4% w/w, and xanthan gum of about 0.2% w/w. In some embodiments, the composition or formulation comprises ammonium acryloyldimethyltaurate/vp copolymer of about 0.3% w/w, a carbomer of about 0.4% w/w, and xanthan gum of about 0.1% w/w. In some embodiments, the viscosity control agents of the composition or formulation comprise ammonium acryloyldimethyltaurate/vp copolymer of about 0.3% w/w, a carbomer of about 0.4% w/w, and xanthan gum of about 0.1% w/w. In some embodiments, the viscosity control agents of the composition or formulation consist essentially of ammonium acryloyldimethyltaurate/vp copolymer of about 0.3% w/w, a carbomer of about 0.4% w/w, and xanthan gum of about 0.1% w/w. In some embodiments, the composition or formulation comprises ammonium acryloyldimethyltaurate/vp copolymer of about 0.3% w/w, a carbomer of about 0.3% w/w, and xanthan gum of about 0.2% w/w. In some embodiments, the viscosity control agents of the composition or formulation comprise ammonium acryloyldimethyltaurate/vp copolymer of about 0.3% w/w, a carbomer of about 0.3% w/w, and xanthan gum of about 0.2% w/w. In some embodiments, the viscosity control agents of the composition or formulation consist essentially of ammonium acryloyldimethyltaurate/vp copolymer of about 0.3% w/w, a carbomer of about 0.3% w/w, and xanthan gum of about 0.2% w/w. In some embodiments, the composition or formulation comprises ammonium acryloyldimethyltaurate/vp copolymer of about 0.4% w/w, a carbomer of about 0.3% w/w, and xanthan gum of about 0.3% w/w. In some embodiments, the viscosity control agents of the composition or formulation comprise ammonium acryloyldimethyltaurate/vp copolymer of about 0.4% w/w, a carbomer of about 0.3% w/w, and xanthan gum of about 0.3% w/w. In some embodiments, the viscosity control agents of the composition or formulation consist essentially of ammonium acryloyldimethyltaurate/vp copolymer of about 0.4% w/w, a carbomer of about 0.3% w/w, and xanthan gum of about 0.3% w/w. In some embodiments, the viscosity control agents of the composition or formulation consist essentially of ammonium acryloyldimethyltaurate/vp copolymer of about 0.4% w/w, a carbomer of about 0.5% w/w, and xanthan gum of about 0.3% w/w. In some embodiments, the viscosity control agents of the composition or formulation consist essentially of ammonium acryloyldimethyltaurate/vp copolymer of about 0.5% w/w, a carbomer of about 0.4% w/w, and xanthan gum of about 0.3% w/w. In some embodiments, the viscosity control agents of the composition or formulation consist essentially of ammonium acryloyldimethyltaurate/vp copolymer of about 0.3% w/w, a carbomer of about 0.6% w/w, and xanthan gum of about 0.3% w/w.

### Skin Conditioners

One or more skin conditioners can be employed in compositions or formulations described herein to improve the appearance and/or conditioning of the skin of the subject following topical application. In some embodiments of compositions or formulations described herein, one or more skin conditioners are present. In some embodiments of compositions or formulations described herein, two or more, three or more, four or more, five or more, six or more, seven or more, eight or more, or nine or more skin conditioners are present. In some embodiments, the one or more skin conditioners comprise a form of hyaluronic acid. In some embodiments, the two or more, three or more, four or more, five or more, six or more, seven or more, eight or more, or nine or more skin conditioners comprise a form of hyaluronic acid. In some embodiments, the form of hyaluronic acid is a salt of hyaluronic acid. In some embodiments, the form of hyaluronic acid is a high molecular weight hyaluronic acid. In some embodiments, the form of hyaluronic acid is sodium hyaluronate. In some embodiments, the form of hyaluronic acid is hydrolyzed hyaluronic acid. In some embodiments, the form of hyaluronic acid is acetylated sodium hyaluronate. In some embodiments, the form of hyaluronic acid is sodium hyaluronate crosspolymer. In some embodiments, the form of hyaluronic acid or sodium hyaluronate reduces oily appearance of the skin of the subject. In some embodiments, the form of hyaluronic acid or sodium hyaluronate improves hydration of the skin, thereby improving skin feel and appearance. Sodium hyaluronate is able to attract and bind moisture to the skin, which helps to reduce skin dryness. In some embodiments, sodium hyaluronate serves to hydrate combination skin, oily skin, and/or acne-prone skin without significantly blocking pores or causing greasiness on the skin surface. In some embodiments, the sodium hyaluronate improves firmness of the skin, thereby improving skin feel and appearance. In some embodiments, the sodium hyaluronate aids in smoothing out fine lines in the skin. In some embodiments, the sodium hyaluronate aids in the reducing the appearance of wrinkles in the skin. In some embodiments, the one or more skin conditioners comprise a natural monocyclic sesquiterpene alcohol. In some embodiments, the two or more, three or more, four or more, five or more, six or more, seven or more, eight or more, or nine or more skin conditioners comprise a natural monocyclic sesquiterpene alcohol. In some embodiments, the natural monocyclic sesquiterpene alcohol is a form of bisabolol. In some embodiments, the bisabolol is α-(−)-bisabolol. In some embodiments, the bisabolol is α-(+)-bisabolol. In some embodiments, the bisabolol is a racemic mixture of the two, α-(±)-bisabolol. Bisabolol is poorly soluble in water and glycerin, but soluble in ethanol. Bisabolol is a colorless viscous oil that may be refined from German chamomile (Matricaria recutita) or Myoporum crassifolium. In some embodiments, bisabolol is synthesized from a biotechnology source. In some embodiments, the form of synthesized bisabolol used in compositions or formulations described herein is Dragosantol® 100. In some embodiments, the form of synthesized bisabolol used in compositions or formulations described herein is about 95% α-bisabolol—specifically, four naturally occurring α-bisabolol isomers—and is almost farnesol free (less than 0.1%). In some embodiments, the bisabolol has skin conditioning properties including aiding in skin repair and healing, reducing the appearance of fine lines and wrinkles, reducing an extent of skin toughness, aiding in the repair of sun-damaged skin, and in improvement of the appearance of acne. In some embodiments, the bisabolol serves as an anti-irritant and reduces skin irritation and redness. In some embodiments, the bisabolol serves as an anti-inflammatory agent and reduces skin inflammation. In some embodiments, the bisabolol serves as an anti-microbial agent and reduces an extent of bacteria present on the skin. In some embodiments, the bisabolol reduces an extent of C. acnes which thereby prevents the accumulation of acne-causing bacteria on the skin of the subject or within pores of the subject. In some embodiments, the bisabolol reduces an extent of Corynebacterium granulosum which thereby prevents the accumulation of acne-causing bacteria on the skin of the subject or within pores of the subject. In some embodiments, the bisabolol reduces an extent of Staphylococcus epidermidis which thereby prevents the accumulation of acne-causing bacteria on the skin of the subject or within pores of the subject. In some embodiments, the bisabolol contributes moisturizing properties to the skin of the subject, thereby improving skin dryness or improving psoriasis. In some embodiments, the bisabolol serves to prevent collagen breakdown in the extracellular matrix of the skin thereby minimizing the appears of fine lines and wrinkles. In some embodiments, the bisabolol serves to reduce redness and irritation, thereby improving the appearance of the skin and the subject's feeling of the skin. In some embodiments, the bisabolol improves rosacea. In some embodiments, the bisabolol reduces discomfort after a sunburn. In some embodiments, the bisabolol improves the appearance of small spots or scars left behind on the skin following the appearance of moderate to severe acne. In some embodiments, the properties of bisabolol aid in clearing acne from the skin of the subject.

In some embodiments, the composition or formulation comprises a form of hyaluronic acid. In some embodiments, the form of hyaluronic acid is selected from sodium hyaluronate, hydrolyzed hyaluronic acid, acetylated sodium hyaluronate, or sodium hyaluronate crosspolymer. In some embodiments, the composition or formulation comprises sodium hyaluronate. In some embodiments, the composition or formulation comprises bisabolol. In some embodiments, the composition or formulation comprises sodium hyaluronate and bisabolol. In some embodiments, the composition or formulation comprises hyaluronic acid and bisabolol. In some embodiments, the composition or formulation comprises hydrolyzed hyaluronic acid and bisabolol. In some embodiments, the composition or formulation comprises acetylated sodium hyaluronate and bisabolol. In some embodiments, the composition or formulation comprises sodium hyaluronate crosspolymer and bisabolol. In some embodiments, the form of hyaluronic acid is present in the composition or formulation at a concentration of at least about 0.001, 0.002, 0.003, 0.004, 0.005, 0.006, 0.007, 0.008, 0.009, 0.01, 0.011, 0.012, 0.013, 0.014, 0.015, 0.02, 0.03, 0.04, 0.05, 0.06, 0.07, 0.08, 0.09, 0.10, 0.11, 0.12, 0.13, 0.14, or 0.15% w/w. In some embodiments, the form of hyaluronic acid is present in the composition or formulation at a concentration of less than about 0.01, 0.011, 0.012, 0.013, 0.014, 0.015, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.25, 0.30, 0.35, or 0.40% w/w. In some embodiments, the form of hyaluronic acid is present in the composition or formulation within the range of about 0.01 to about 0.1% w/w. In some embodiments, the form of hyaluronic acid is present in the composition or formulation at a concentration of about 0.01% w/w. In some embodiments, the form of hyaluronic acid is present in the composition or formulation at a concentration of about 0.05% w/w. In some embodiments, the form of hyaluronic acid is present in the composition or formulation at a concentration of about 0.10% w/w. In some embodiments, sodium hyaluronate is present in the composition or formulation at a concentration of about 0.01% w/w. In some embodiments, sodium hyaluronate is present in the composition or formulation at a concentration of about 0.025% w/w. In some embodiments, sodium hyaluronate is present in the composition or formulation at a concentration of about 0.05% w/w. In some embodiments, sodium hyaluronate is present in the composition or formulation at a concentration of about 0.075% w/w. In some embodiments, sodium hyaluronate is present in the composition or formulation at a concentration of about 0.1% w/w. In some embodiments, the bisabolol is present in the composition or formulation at a concentration of at least about 0.001, 0.002, 0.003, 0.004, 0.005, 0.006, 0.007, 0.008, 0.009, 0.01, 0.011, 0.012, 0.013, 0.014, 0.015, 0.02, 0.03, 0.04, 0.05, 0.06, 0.07, 0.08, 0.09, 0.10, 0.11, 0.12, 0.13, 0.14, or 0.15% w/w. In some embodiments, the bisabolol is present in the composition or formulation at a concentration of less than about 0.01, 0.011, 0.012, 0.013, 0.014, 0.015, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.25, 0.30, 0.35, 0.40, 0.45, or 0.50% w/w. In some embodiments, the bisabolol is present in the composition or formulation within the range of about 0.001 to about 0.1% w/w. In some embodiments, the bisabolol is present in the composition or formulation at a concentration of about 0.001% w/w. In some embodiments, the bisabolol is present in the composition or formulation at a concentration of about 0.005% w/w. In some embodiments, the bisabolol is present in the composition or formulation at a concentration of about 0.01% w/w. In some embodiments, the bisabolol is present in the composition or formulation at a concentration of about 0.02% w/w. In some embodiments, the bisabolol is present in the composition or formulation at a concentration of about 0.05% w/w. In some embodiments, the bisabolol is present in the composition or formulation at a concentration of about 0.10% w/w. In some embodiments, the bisabolol is present in the composition or formulation at a concentration of about 0.20% w/w. In some embodiments, the bisabolol is present in the composition or formulation at a concentration of about 0.30% w/w. In some embodiments, the bisabolol is present in the composition or formulation at a concentration of about 0.40% w/w. In some embodiments, the bisabolol is present in the composition or formulation at a concentration of about 0.50% w/w. In some embodiments, the composition or formulation comprises sodium hyaluronate present within the range of about 0.001 to about 0.1% w/w and bisabolol present within the range of about 0.001 to about 0.5% w/w.

### Subjects

In some instances, compositions and formulations described herein are used as part of a skin care regimen that comprises topical application of the composition or formulation. In some embodiments of skin care regimen, the skin care regimen is designed to improve or clear acne in the subject. In some instances, the subject is assessed to determine their skin type (e.g., normal, dry, oily, combination, sensitive, or the like), acne type and acne severity, Fitzpatrick skin complexion type, or other skin conditions and concerns to inform parameters of the skin care regimen, or any combination thereof. The formulation used and frequency of use may vary with the skin assessment of the subject, the age and physical condition of the subject, the duration of the treatment, the response and tolerability by the subject, and like factors. In some instances, the subject is a mammal. In some instances, the subject is a human. In some instances, the subject is a male. In some instances, the subject is a female. In some instances, the human subject is at least about 18 years of age. In some instances, the human subject is a child or adult 12 years of age or over. In some instances, the human subject is an adult between the ages of 18-79.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity to whom the composition or formulation is applied. subject can be a human. The subject may be diagnosed or observed to have a dermatological condition. The dermatological condition may include acne vulgaris.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

### Examples

The following examples are included for illustrative purposes only and are not intended to limit the scope of the inventive concepts,

### Example 1: Quantitative Advanced Formulation of 2.5% Micronized Benzoyl Peroxide for Use with Acne Home Care Protocol

In this example, an advanced acne medication formula is presented for use in home skin care protocol for acne improvement and clearance. Key ingredients of the formulation include about 2.5% micronized benzoyl peroxide, bisabolol, and sodium hyaluronate. Micronized benzoyl peroxide is utilized for a smaller and more consistent benzoyl peroxide particle size that penetrates skin pores more efficiently and has a smoother, less gritty consistency than non-micronized benzoyl peroxide. This micronized benzoyl peroxide also prevents potential skin irritation following topical application to the face of a subject in need thereof. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The smoother consistency and the less potential for dryness and/or irritation contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness.

### Contraindications

This formulation is not to be provided to subjects that are allergic to benzoyl peroxide.

The components and respective concentrations in the formulation are listed in Table 1 below. As provided in Table 1, the International Nomenclature of Cosmetic Ingredients (INCI)/Common Name is provided for each component.

TABLE 1
Formula A - Acne Medication 2.5% BPO

| INCI/Common Name | % w/W |
|---|---|
| Benzoyl Peroxide | 2.5 - 2.625 |
| Glycerin | 2.991 |
| Phenoxyethanol | 0.900 |
| Ethylhexylglycerin | 0.100 |
| Propanediol | 1.000 |
| Sodium Hydroxide | 0.160 |
| Carbomer | 0.400 |
| Ammonium | 0.300 |
| Acryloyldimethyltaurate/VP | |
| Copolymer | |
| Xanthan Gum | 0.100 |
| Sodium Hyaluronate | 0.01-0.1 |
| Bisabolol | 0.010 - 0.10 |
| Aqua | QS 100 |

### Example 2: Quantitative Advanced Formulation of 2.5% Micronized Benzoyl Peroxide for Use with Acne Home Care Protocol with Sodium Hyaluronate or Sodium Hyaluronate Crosspolymer

In this example, an advanced acne medication formula is presented for use in home skin care protocol for acne improvement and clearance. Key ingredients of the formulation include about 2.5% micronized benzoyl peroxide, bisabolol, and sodium hyaluronate. Micronized benzoyl peroxide is utilized for a smaller and more consistent benzoyl peroxide particle size that penetrates skin pores more efficiently and has a smoother, less gritty consistency than non-micronized benzoyl peroxide. This micronized benzoyl peroxide also prevents potential skin irritation following topical application to the face of a subject needing acne clearance. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The less potential for dryness and/or irritation and smoother consistency contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness.

### Contraindications

This formulation is not to be provided to subjects that are allergic to benzoyl peroxide.

The components and respective concentrations in the formulation are listed in Table 2 below. As provided in Table 2, the International Nomenclature of Cosmetic Ingredients (INCI)/Common Name is provided for each component.

TABLE 2
Formula B - Acne Medication 2.5% BPO

| INCI/Common Name | % w/w |
|---|---|
| Benzoyl Peroxide | 2.5 - 2.625 |
| Glycerin | 2.49 |
| Phenoxyethanol | 0.800 |
| Ethylhexylglycerin | 0.200 |
| Propanediol | 1.000 |
| Sodium Hydroxide | 0.100 |
| Carbomer | 0.300 |
| Ammonium | 0.300 |
| Acryloyldimethyltaurate/VP | |
| Copolymer | |
| Xanthan Gum | 0.200 |
| Sodium Hyaluronate or | 0.01-0.1 |
| sodium hyaluronate | |
| crosspolymer | |
| Bisabolol | 0.01 - 0.50 |
| Aqua | QS 100 |

### Example 3: Quantitative Advanced Formulation of 5% Micronized Benzoyl Peroxide for Use with Acne Home Care Protocol

In this example, an advanced acne medication formula is presented for use in home skin care protocol for acne improvement and clearance. Key ingredients of the formulation include about 5% micronized benzoyl peroxide, bisabolol, and sodium hyaluronate. Micronized benzoyl peroxide is utilized for a smaller and more consistent benzoyl peroxide particle size that penetrates skin pores more efficiently and has a smoother, less gritty consistency than non-micronized benzoyl peroxide. This micronized benzoyl peroxide also prevents potential skin irritation following topical application to the face of a subject needing acne clearance. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The less potential for dryness and/or irritation and smoother consistency contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness.

### Contraindications

This formulation is not to be provided to subjects that are allergic to benzoyl peroxide.

The components and respective concentrations in the formulation are listed in Table 3 below. As provided in Table 3, the International Nomenclature of Cosmetic Ingredients (INCI)/Common Name is provided for each component.

TABLE 3
Formula C - Acne Medication 5% BPO

| INCI/Common Name | % w/w |
|---|---|
| Benzoyl Peroxide | 5.0 - 5.25 |
| Glycerin | 2.991 |
| Phenoxyethanol | 0.900 |
| Ethylhexylglycerin | 0.100 |
| Propanediol | 1.000 |
| Sodium Hydroxide | 0.100 |
| Carbomer | 0.400 |
| Ammonium | 0.400 |
| Acryloyldimethyltaurate/VP | |
| Copolymer | |
| Xanthan Gum | 0.200 |
| Sodium Hyaluronate | 0.01-0.1 |
| Bisabolol | 0.010 - 0.10 |
| Aqua | QS 100 |

### Example 4: Quantitative Advanced Formulation of 5% Micronized Benzoyl Peroxide for Use with Acne Home Care Protocol with Sodium Hyaluronate or Sodium Hyaluronate Crosspolymer

In this example, an advanced acne medication formula is presented for use in home skin care protocol for acne improvement and clearance. Key ingredients of the formulation include about 5% micronized benzoyl peroxide, bisabolol, and sodium hyaluronate. Micronized benzoyl peroxide is utilized for a smaller and more consistent benzoyl peroxide particle size that penetrates skin pores more efficiently and has a smoother, less gritty consistency than non-micronized benzoyl peroxide. This micronized benzoyl peroxide also prevents potential skin irritation following topical application to the face of a subject needing acne clearance. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The less potential for dryness and/or irritation and smoother consistency contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness.

### Contraindications

This formulation is not to be provided to subjects that are allergic to benzoyl peroxide.

The components and respective concentrations in the formulation are listed in Table 4 below. As provided in Table 4, the International Nomenclature of Cosmetic Ingredients (INCI)/Common Name is provided for each component.

TABLE 4
Formula D - Acne Medication 5% BPO

| INCI/Common Name | % w/w |
|---|---|
| Benzoyl Peroxide | 5.0 - 5.25 |
| Glycerin | 2.49 |
| Phenoxyethanol | 0.800 |
| Ethylhexylglycerin | 0.200 |
| Propanediol | 1.000 |
| Sodium Hydroxide | 0.100 |
| Carbomer | 0.300 |
| Ammonium | 0.400 |
| Acryloyldimethyltaurate/VP | |
| Copolymer | |
| Xanthan Gum | 0.300 |
| Sodium Hyaluronate or | 0.01-0.1 |
| sodium hyaluronate | |
| crosspolymer | |
| Bisabolol | 0.001 - 0.1 |
| Aqua | QS 100 |

### Example 5: Quantitative Advanced Formulation of 10% Micronized Benzoyl Peroxide for Use with Acne Home Care Protocol

In this example, an advanced acne medication formula is presented for use in home skin care protocol for acne improvement and clearance. Key ingredients of the formulation include about 10% micronized benzoyl peroxide, bisabolol, and sodium hyaluronate. Micronized benzoyl peroxide is utilized for a smaller and more consistent benzoyl peroxide particle size that penetrates skin pores more efficiently than non-micronized benzoyl peroxide. This micronized benzoyl peroxide also prevents potential skin irritation following topical application to the face of a subject needing acne clearance. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The less potential for dryness and/or irritation contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness.

### Contraindications

This formulation is not to be provided to subjects that are allergic to benzoyl peroxide.

The components and respective concentrations in the formulation are listed in Table 5 below. As provided in Table 5, the International Nomenclature of Cosmetic Ingredients (INCI)/Common Name is provided for each component.

TABLE 5
Formula E - Acne Medication 10% BPO

| INCI/Common Name | $% w/W |
|---|---|
| Benzoyl Peroxide | 10.0 - 10.5 |
| Glycerin | 2.991 |
| Phenoxyethanol | 0.900 |
| Ethylhexylglycerin | 0.100 |
| Propanediol | 1.000 |
| Sodium Hydroxide | 0.160 |
| Carbomer | 0.500 |
| Ammonium | 0.400 |
| Acryloyldimethyltaurate/VP | |
| Copolymer | |
| Xanthan Gum | 0.300 |
| Sodium Hyaluronate | 0.01-0.1 |
| Bisabolol | 0.010 - 0.10 |
| Aqua | QS 100 |

### Example 6: Quantitative Advanced Formulation of 10% Micronized Benzoyl Peroxide for Use with Acne Home Care Protocol with Sodium Hyaluronate or Sodium Hyaluronate Crosspolymer

In this example, an advanced acne medication formula is presented for use in home skin care protocol for acne improvement and clearance. Key ingredients of the formulation include about 10% micronized benzoyl peroxide, bisabolol, and sodium hyaluronate. Micronized benzoyl peroxide is utilized for a smaller and more consistent benzoyl peroxide particle size that penetrates skin pores more efficiently than non-micronized benzoyl peroxide. This micronized benzoyl peroxide also prevents potential skin irritation following topical application to the face of a subject needing acne clearance. Micronized benzoyl peroxide is effective as an antimicrobial agent, capable of effectively killing Cutibacterium acnes (C. acnes). C. acnes is a relatively slow growing, typically aerotolerant anaerobic, gram-positive bacterium (rod) linked to the skin condition of acne. The formulation described herein effectively kills C. acnes and aids in peeling the inside of the pore of the skin. The formulation in this example offers an effective topical solution for use in clearance of mild to moderate acne vulgaris with less potential for dryness and/or irritation following topical application. The less potential for dryness and/or irritation contributes to more successful compliance by the subject with a skin care regimen including daily topical application of the advanced acne medication formula presented herein. Bisabolol in the formulation supports skin hydration, skin soothing, reduces skin irritation, and reduces skin sensitivity. Sodium hyaluronate in the formulation boosts skin resilience in the subject undergoing topical application, and attracts moisture to the skin, which can help reduce dryness and flakiness.

### Contraindications

This formulation is not to be provided to subjects that are allergic to benzoyl peroxide.

The components and respective concentrations in the formulation are listed in Table 6 below. As provided in Table 6, the International Nomenclature of Cosmetic Ingredients (INCI)/Common Name is provided for each component.

TABLE 6
Formula F - Acne Medication 10% BPO

| INCI/Common Name | % w/W |
|---|---|
| Benzoyl Peroxide | 10.0 - 10.5 |
| Glycerin | 2.49 |
| Phenoxyethanol | 0.800 |
| Ethylhexylglycerin | 0.200 |
| Propanediol | 1.000 |
| Sodium Hydroxide | 0.100 |
| Carbomer | 0.600 |
| Ammonium | 0,300 |
| Acryloyldimethyltaurate/VP | |
| Copolymer | |
| Xanthan Gum | 0.300 |
| Sodium Hyaluronate or | 0.01-0.25 |
| sodium hyaluronate | |
| crosspolymer | |
| Bisabolol | 0.010 - 0.50 |
| Aqua | QS 100 |

### Example 7: Repeated Insult Patch Test to Assess Tolerability of Advanced Acne Medication Formulations

Human repeated insult patch tests (HRIPT) were conducted to confirm, in healthy human subjects, that a test product was not associated with a dermal sensitization (allergic) response under repeated semi-occlusive patch test conditions. The methodology was also used to assess skin irritation during the Induction phase of the test.

### Test Subjects

At least 50 male and female subjects ranging in ages 18 to 79 years were empaneled for each test. The subjects chosen were able to read and understand instructions. The subjects did not exhibit any physical or dermatologic condition that would have precluded application of the test article or determination of potential effects of the test article.

### Test Articles

Advanced acne medication formulations described herein were tested in the HRIPT assay of this example. Test articles of advanced acne medication formulations tested in the HRIPT assay were formulated comprising a micronized benzoyl peroxide anti-acne agent, a moisturizing agent, one or more solvents, one or more preservatives, a pH adjuster, one or more viscosity control agents, and at least two skin conditioners. Bisabolol is one of the skin conditioners in each test article.

### Test Procedure

The 9 Repeated Insult (semi-occlusive) Patch Test (9-RIPT) was conducted as follows.

### Induction Phase

A sufficient amount of the test article (an amount to adequately cover the surface of the patch unit-approximately 0.1 g-0.15 g) was placed onto an approximate 2 cm×2 cm square of Webril® cotton fabric (approximately 25-38 mg/cm² of test material) affixed to Scanpor (Allerderm) semi-occlusive surgical tape. The patch was applied to an application site the back of each subject between the scapulae and waist, adjacent to the spinal mid-line. This application procedure was performed by a trained technician/examiner at a Testing Facility and repeated every Monday, Wednesday, and Friday until 9 applications of the test article had been made on the subjects. The subjects were instructed to remove the patch 24 hours after application. Twenty-four hour rest periods followed the Tuesday and Thursday removals and 48-hour rest periods followed each Saturday removal. Subjects returned to the Testing Facility and the application site was scored by a trained examiner just prior to the next patch application. If a subject developed a positive reaction of a level 2 erythema or greater during the Induction phase or if, at the discretion of the Study Director, the skin response warranted a change in application site, the patch was applied to a previously unpatched, adjacent application site for the next application. If a level 2 reaction or greater occurred at the new site, no further applications were made on the subject. However, any reactive subjects were subsequently Challenge patch tested.

### Challenge Phase

After a rest period of approximately 2 weeks (no applications of the test article), the Challenge patch was applied to a previously unpatched (virgin) test site. A Challenge patch refers to a patch containing a test article applied to a previously unpatched area of the subject's skin following the rest period. A Challenge patch is used to assess if the subject's skin has become sensitized to the test article by observing any reactions at that new site. Reactions observed at site of Challenge patch may confirm the presence of any potential allergic reactions after exposure to the test article during the induction phase. The subjects returned to the Testing Facility 24 hours after the Challenge patch application and the Challenge patch was removed. The site was scored immediately after patch removal (the first score). Subjects returned to the Testing Facility 72 hours after application (48 hours after removal of the Challenge patch) and the site was scored again (the second score). All subjects were instructed to report any delayed skin reactivity that occurred after the second Challenge patch score. When warranted, selected test subjects were called back to the Clinic for additional examinations and scoring to determine possible increases or decreases in Challenge patch reactivity.

### Scoring of Dermal Responses

Dermal responses for both the Induction and Challenge phases of the study were scored according to the following 6-point scale:
0=No evidence of any effect
+=Barely perceptible (Minimal, faint, uniform or spotty erythema)
1=Mild (Pink, uniform erythema covering most of the contact site)
2=Moderate (Pink-red erythema uniform in the entire contact site)
3=Marked (Bright red erythema with/without petechiae or papules)
4=Severe (Deep red erythema with/without vesiculation or weeping)

All other observed dermal sequelae (e.g., edema, dryness, hypo- or hyperpigmentation) were appropriately recorded on the data sheet and described as mild, moderate, or severe.

### Data Interpretation

Edema, vesicles, papules and/or erythema that persist or increase in intensity either during the Induction and/or Challenge phase may be indicative of allergic contact dermatitis. Allergic responses normally do not resolve or improve markedly at 72-96 hours.

Exceptions to typical skin reactions may occur. These may include, but not be limited to, symptoms of allergic contact sensitivity early in the Induction period to one or more test products. When this occurs in one subject, such a reaction usually suggests either an idiosyncratic response or that the subject had a pre-exposure/sensitization to the test material or component(s) of the test material or a cross-reactivity with a similar product/component. Data for such reactions will be included in the study report but will not be included in the final study analysis/conclusion of sensitization.

### Results

For each of test articles of Formula A, Formula C, and Formula E (listed in Examples 1, 3, and 5 respectively), a total of 55 subjects (15 male subjects and 40 female subjects ranging in age from 18 to 75 years) were empaneled for the test procedure. Fifty (50 out of 55 subjects) subjects satisfactorily completed the test procedure for each test article. There was no skin reactivity observed at any time during the course of each study. Under the conditions of a repeated insult (semi-occlusive) patch test procedure conducted in 50 subjects, test article Formula A, Formula C, and Formula E were each not associated with skin irritation or allergic contact dermatitis in human subject. All test articles passed with no erythema.

While preferred aspects of the present inventive concepts have been shown and described herein, it will be obvious to those skilled in the art that such aspects are provided by way of example only. Numerous variations, changes, and substitutions will now occur to those skilled in the art without departing from the inventive concepts. It should be understood that various alternatives to the aspects of the inventive concepts described herein may be employed in practicing the inventive concepts. It is intended that the following claims define the scope of the inventive concepts and that methods and structures within the scope of these claims and their equivalents be covered thereby.

### Aspects

Aspect 1. A composition comprising:
i) micronized benzoyl peroxide at a concentration of between about 2.5% w/w to about 10.5% w/w;
ii) sodium hyaluronate at a concentration of between about 0.001- to about 0.5%; and
iii) bisabolol at a concentration of between about 0.001- to about 0.5%.

Aspect 2. The composition of aspect 1, further comprising one or more humectants.

Aspect 3. The composition of aspect 2, wherein the one or more humectants comprise glycerin, or ethylhexylglycerin, or a combination thereof.

Aspect 4. The composition of any one of aspects 1-3, further comprising one or more preservatives.

Aspect 5. The composition of aspect 4, wherein the one or more preservatives comprise phenoxyethanol, or ethylhexylglycerin, or a combination thereof.

Aspect 6. The composition of any one of aspects 1-5, further comprising one or more solvents.

Aspect 7. The composition of aspect 6, wherein the one or more solvents comprise aqua.

Aspect 8. The composition of aspect 7, wherein the one or more solvents further comprise propanediol.

Aspect 9. The composition of any one of aspects 1-8, further comprising one or more buffering agents.

Aspect 10. The composition of aspect 9, wherein the one or more buffering agents comprise sodium hydroxide.

Aspect 11. The composition of any one of aspects 1-10, further comprising one of more viscosity control agents.

Aspect 12. The composition of aspect 11, wherein the one or more viscosity control agents comprise a carbomer, ammonium acryloyldimethyltaurate/VP copolymer, or xanthan gum, or any combination thereof.

Aspect 13. The composition of aspect 11, wherein the one or more viscosity control agents comprise a carbomer, ammonium acryloyldimethyltaurate/VP copolymer, and xanthan gum.

Aspect 14. The composition of any one of aspects 7-13, wherein the aqua comprises less than about 92% w/w of the composition.

Aspect 15. The composition of any one of aspects 1-14, wherein the micronized benzoyl peroxide is at a concentration of about 2.5% w/v.

Aspect 16. The composition of any one of aspects 1-14, wherein the micronized benzoyl peroxide is at a concentration of about 5% w/v.

Aspect 17. The composition of any one of aspects 1-14, wherein the micronized benzoyl peroxide is at a concentration of about 10% w/v.

Aspect 18. The composition of any one of aspects 1-17, wherein the sodium hyaluronate is at a concentration of about 0.1%.

Aspect 19. The composition of any one of aspects 1-17, wherein the sodium hyaluronate is at a concentration of about 0.01%.

Aspect 20. The composition of any one of aspects 1-19, wherein the bisabolol is at a concentration of about 0.1%.

Aspect 21. The composition of any one of aspects 1-19, wherein the bisabolol is at a concentration of about 0.01%.

Aspect 22. An acne clearance formulation comprising:
i) micronized benzoyl peroxide at a concentration of between about 2.5% w/w to about 10.5% w/w;
ii) sodium hyaluronate at a concentration of between about 0.001- to about 0.5%; and
iii) bisabolol at a concentration of between about 0.001- to about 0.5%;
   wherein the acne clearance formulation is formulated as a cream, gel, gel-cream, lotion, solution, emulsion, suspension or serum, for topical application to facial skin of a subject in need thereof.

Aspect 23. The formulation of aspect 22, further comprising one or more humectants.

Aspect 24. The composition of aspect 23, wherein the one or more humectants comprise glycerin, or ethylhexylglycerin, or a combination thereof.

Aspect 25. The formulation of any one of aspects 22-24, further comprising one or more preservatives.

Aspect 26. The formulation of aspect 25, wherein the one or more preservatives comprise phenoxyethanol, or ethylhexylglycerin, or a combination thereof.

Aspect 27. The formulation of any one of aspects 22-26, further comprising one or more solvents.

Aspect 28. The formulation of aspect 27, wherein the one or more solvents comprise aqua.

Aspect 29. The formulation of aspect 28, wherein the one or more solvents further comprise propanediol.

Aspect 30. The formulation of any one of aspects 22-29, further comprising one or more buffering agents.

Aspect 31. The formulation of aspect 30, wherein the one or more buffering agents comprises sodium hydroxide.

Aspect 32. The formulation of any one of aspects 22-31, further comprising one of more viscosity control agents.

Aspect 33. The formulation of aspect 32, wherein the one or more viscosity control agents comprise a carbomer, ammonium acryloyldimethyltaurate/VP copolymer, or xanthan gum, or any combination thereof.

Aspect 34. The formulation of aspect 32, wherein the one or more viscosity control agents comprise a carbomer, ammonium acryloyldimethyltaurate/VP copolymer, and xanthan gum.

Aspect 35. The formulation of any one of aspects 28-33, wherein the aqua comprises less than about 92% w/w of the formulation.

Aspect 36. The formulation of any one of aspects 22-35, wherein the micronized benzoyl peroxide is at a concentration of about 2.5% w/w.

Aspect 37. The formulation of any one of aspects 22-35, wherein the micronized benzoyl peroxide is at a concentration of about 2.625% w/w.

Aspect 38. The formulation of any one of aspects 22-35, wherein the micronized benzoyl peroxide is at a concentration of about 4.0% w/w.

Aspect 39. The formulation of any one of aspects 22-35, wherein the micronized benzoyl peroxide is at a concentration of about 5.0% w/w.

Aspect 40. The formulation of any one of aspects 22-35, wherein the micronized benzoyl peroxide is at a concentration of about 5.25% w/w.

Aspect 41. The formulation of any one of aspects 22-35, wherein the micronized benzoyl peroxide is at a concentration of about 9.0% w/w.

Aspect 42. The formulation of any one of aspects 22-35, wherein the micronized benzoyl peroxide is at a concentration of about 10.0% w/w.

Aspect 43. The formulation of any one of aspects 22-35, wherein the micronized benzoyl peroxide is at a concentration of about 10.5% w/w.

Aspect 44. The formulation of any one of aspects 22-43, wherein a Doo of particle size of the micronized benzoyl peroxide is between about 0.35 to 15 microns.

Aspect 45. The formulation of any one of aspects 22-44, wherein a particle size of the micronized benzoyl peroxide is no greater than 25 microns.

Aspect 46. The formulation of any one of aspects 22-45, wherein the sodium hyaluronate is at a concentration of at least about 0.005%.

Aspect 47. The formulation of any one of aspects 22-46, wherein the sodium hyaluronate is at a concentration of about 0.01%.

Aspect 48. The formulation of any one of aspects 22-46, wherein the sodium hyaluronate is at a concentration of about 0.05%.

Aspect 49. The formulation of any one of aspects 22-46, wherein the sodium hyaluronate is at a concentration of about 0.1%.

Aspect 50. The formulation of any one of aspects 22-49, wherein the bisabolol is at a concentration of at least about 0.005%.

Aspect 51. The formulation of any one of aspects 22-50, wherein the bisabolol is at a concentration of at about 0.01%

Aspect 52. The formulation of any one of aspects 22-50, wherein the bisabolol is at a concentration of at least about 0.05%.

Aspect 53. The formulation of any one of aspects 22-50, wherein the bisabolol is at a concentration of at least about 0.1%.

Aspect 54. The formulation of aspect 35, formulated as an aqueous solution,
wherein the micronized benzoyl peroxide is at a concentration of about 2.625% w/w;
wherein the sodium hyaluronate is at a concentration of about 0.1%;
wherein the bisabolol is at a concentration of about 0.01%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

Aspect 55. The formulation of aspect 35, formulated as an aqueous solution,
wherein the micronized benzoyl peroxide is at a concentration of about 2.625% w/w;
wherein the sodium hyaluronate is at a concentration of about 0.01%;
wherein the bisabolol is at a concentration of about 0.1%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

Aspect 56. The formulation of aspect 35, formulated as an aqueous solution,
wherein the micronized benzoyl peroxide is at a concentration of about 5.25% w/w;
wherein the sodium hyaluronate is at a concentration of about 0.01%;
wherein the bisabolol is at a concentration of about 0.01%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

Aspect 57. The formulation of aspect 35, formulated as an aqueous solution,
wherein the micronized benzoyl peroxide is at a concentration of about 5.25% w/w;
wherein the sodium hyaluronate is at a concentration of about 0.1%;
wherein the bisabolol is at a concentration of about 0.1%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

Aspect 58. The formulation of aspect 35, formulated as an aqueous solution,
wherein the micronized benzoyl peroxide is at a concentration of about 10.5% w/w;
wherein the sodium hyaluronate is at a concentration of about 0.01%;
wherein the bisabolol is at a concentration of about 0.01%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

Aspect 59. The formulation of aspect 35, formulated as an aqueous solution,
wherein the micronized benzoyl peroxide is at a concentration of about 10.5% w/w;
wherein the sodium hyaluronate is at a concentration of about 0.1%;
wherein the bisabolol is at a concentration of about 0.1%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

What is claimed is:

1. A composition comprising:
i) micronized benzoyl peroxide at a concentration of between about 2.5% w/w to about 10.5% w/w;
ii) sodium hyaluronate at a concentration of about 0.001% to about 0.1%; and
iii) bisabolol at a concentration of about 0.001% to about 0.5%.

2. The composition of claim 1, further comprising one or more humectants, wherein the one or more humectants comprise glycerin, or ethylhexylglycerin, or a combination thereof.

3. The composition of claim 1, further comprising one or more preservatives, wherein the one or more preservatives comprise phenoxyethanol, or ethylhexylglycerin, or a combination thereof.

4. The composition of claim 1, further comprising one or more solvents, wherein the one or more solvents comprises aqua, propanediol, or aqua and propanediol.

5. The composition of claim 1, further comprising sodium hydroxide.

6. The composition of claim 1, further comprising one of more viscosity control agents, wherein the one or more viscosity control agents comprise a carbomer, ammonium acryloyldimethyltaurate/VP copolymer, or xanthan gum, or any combination thereof.

7. The composition of claim 1, wherein the micronized benzoyl peroxide is at a concentration of about 2.625% w/w, about 5.25% w/w, or about 10.5% w/w.

8. The composition of claim 1, wherein the sodium hyaluronate is at a concentration of about 0.01% or about 0.1%.

9. The composition of claim 1, wherein the bisabolol is at a concentration of about 0.01% or about 0.1%.

10. An acne clearance formulation comprising:
i) micronized benzoyl peroxide at a concentration of between about 2.5% w/w to about 10.5% w/w;
ii) sodium hyaluronate at a concentration of between about 0.001- to about 0.1%; and
iii) bisabolol at a concentration of between about 0.001- to about 0.5%;
wherein the acne clearance formulation is formulated as a cream, a gel, a gel-cream, a lotion, a solution, an emulsion, a suspension, or a serum, for topical application to facial skin of a subject in need thereof.

11. The formulation of claim 10, further comprising one or more humectants, wherein the one or more humectants comprise glycerin, or ethylhexylglycerin, or a combination thereof.

12. The composition of claim 10, further comprising one or more preservatives, wherein the one or more preservatives comprise phenoxyethanol, or ethylhexylglycerin, or a combination thereof.

13. The formulation of claim 10, further comprising one or more solvents, wherein the one or more solvents comprises aqua, propanediol, or aqua and propanediol.

14. The formulation of claim 10, further comprising sodium hydroxide.

15. The formulation of claim 10, further comprising one of more viscosity control agents, wherein the one or more viscosity control agents comprise a carbomer, ammonium acryloyldimethyltaurate/VP copolymer, or xanthan gum, or any combination thereof.

16. The formulation of claim 10, wherein the micronized benzoyl peroxide is at a concentration of about 2.625% w/w, of about 5.25% w/w, or about 10.5% w/w.

17. The formulation of claim 16, wherein the sodium hyaluronate is at a concentration of about 0.01% or about 0.1%.

18. The formulation of claim 17, wherein the bisabolol is at a concentration of about 0.01% or about 0.1%.

19. The formulation of claim 10, wherein the formulation is formulated as an aqueous solution,
wherein the micronized benzoyl peroxide is at a concentration of about 2.625% w/w;
wherein the sodium hyaluronate is at a concentration of about 0.1%;
wherein the bisabolol is at a concentration of about 0.01%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

20. The formulation of claim 10, wherein the formulation is formulated as an aqueous solution,
wherein the micronized benzoyl peroxide is at a concentration of about 5.25% w/w;
wherein the sodium hyaluronate is at a concentration of about 0.01%;
wherein the bisabolol is at a concentration of about 0.01%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

21. The formulation of claim 10, wherein the formulation is formulated as an aqueous solution,
wherein the micronized benzoyl peroxide is at a concentration of about 10.5% w/w;
wherein the sodium hyaluronate is at a concentration of about 0.01%;
wherein the bisabolol is at a concentration of about 0.01%;
wherein the formulation further comprises inactive ingredients selected from the group consisting of aqua, glycerin, propanediol, a carbomer, ammonium acryloyldimethyltaurate/VP copolymer, xanthan gum, sodium hydroxide, phenoxyethanol, and ethylhexylglycerin; and
wherein the formulation is effective at clearing acne or acne rosacea when topically applied as part of a skin care regimen.

22. An acne clearance formulation comprising:
i) micronized benzoyl peroxide at a concentration of between about 2.5% to about 10.5%, wherein the micronized benzoyl peroxide has a particle size of no greater than about 25 microns;
ii) a form of hyaluronic acid selected from hydrolyzed hyaluronic acid, sodium hyaluronate, sodium hyaluronate crosspolymer, or acetylated sodium hyaluronate, wherein the form of hyaluronic acid is at a concentration of between about 0.01- to about 0.1%;
iii) bisabolol at a concentration of between about 0.01- to about 0.25%;
iv) two or more humectants, wherein the two or more humectants comprise butylene glycol, cyclomethicone, dipropylene ethylhexylglycerin, glycol, glycerin, propylene glycol, sodium lactate, sorbitol, trehalose, xylitol, or sorbitol, or any combination thereof;
v) two or more solvents;
vi) one or more viscosity control agents;
vii) a buffering agent; and
viii) a preservative.
